(19) United States
(12) Patent Application Publication
(10) Pub. No.: US 2014/0121163 A1
(43) Pub. Date: May 1, 2014

(54) MFG-E8 AND USES THEREOF

(71) Applicant: Ping Wang, Roslyn, NY (US)

(72) Inventor: Ping Wang, Roslyn, NY (US)

(73) Assignee: The Feinstein Institute for Medical Research a corporation

(21) Appl. No.: 14/114,397

(22) PCT Filed: Apr. 27, 2012
(86) PCT No.: PCT/US2012/035362

§ 371 Date: Dec. 17, 2013

Related U.S. Application Data
(60) Provisional application No. 61/480,031, filed on Apr. 28, 2011.

(51) Int. Cl. A61K 38/18

(52) U.S. Cl. CPC A61K 38/1808
USPC 514/9.6

(57) ABSTRACT

Methods of treating cerebral ischemia using milk fat globule epidermal growth factor-factor VIII (MFG-E8) are disclosed, as are recombinant human MFG-E8 and its uses in pharmaceutical compositions, products and methods for treating inflammation and organ injury after ischemia/reperfusion, sepsis, and lung injury.

### CROSS-REFERENCE TO RELATED APPLICATIONS

This application claims the benefit of PCT Application No. PCT/US2012/35362, filed Apr. 27, 2012, which claims the benefit of U.S. Provisional Patent Application No. 61/480,031, filed Apr. 28, 2011, the content of which is herein incorporated by reference in its entirety.

### STATEMENT OF GOVERNMENT SUPPORT

This invention was made with government support under grant number GM057468 awarded by the National Institutes of Health. The government has certain rights in the invention.

### BACKGROUND OF THE INVENTION

Throughout this application various publications are referred to in parentheses. Full citations for these references may be found at the end of the specification. The disclosures of these publications are hereby incorporated by reference in their entirety into the subject application to more fully describe the art to which the subject invention pertains.

Inflammation and apoptosis play crucial roles in the evolution of cerebral infarct following ischemic insult. Following necrotic cell death in the core of the cerebral infarct, cell death in the relatively hypoperfused penumbra occurs over time through inflammatory and apoptotic mechanisms. The inflammatory process involves NF-κB mediated release of cytokines, such as TNF-α, which cause cell injury (Dirnagl et al. 1999). Apoptosis involves release of pro-apoptotic molecules such as bax, and activation of the caspases leading to DNA fragmentation and cell death. The cell-damaging mechanisms that are activated by ischemia are countered by cell-survival mechanisms including upregulation of anti-apoptotic molecules such as bcl-2 (Antonsson 2004). The peroxisome-proliferator activated receptor-γ (PPAR-γ) is a ligand-inducible transcription factor that has been shown to counteract inflammation by downregulating cytokine release (Ricote and Glass 2007). Therapeutic suppression of inflammation and apoptosis could rescue the penumbra after ischemic stroke.

Sepsis is one of the most prevalent diseases and accounts for 20% of all admissions to intensive care units (ICUs) (Angus, et al., 2001). Evidence indicates that in the U.S. alone, more than 750,000 people develop sepsis each year with an overall mortality rate of 28.6% (Angus, et al., 2001). Despite advances in the management of septic patients, a large number of such patients die of the ensuing septic shock and multiple organ failure (Ferrer, et al., 2008; Strehlow, et al., 2006; Martin, et al., 2003; Guidet, et al., 2005). An analysis of hospital records indicates that the total number of patients who have died of sepsis is actually increasing (Martin, et al., 2003). As the American population ages, the incidence of sepsis is projected to increase since the incidence and mortality rate of sepsis rise steadily with aging (Angus, et al., 2001; Martin, et al., 2003). Thus, there is an urgent unmet medical need for an effective novel therapy for patients with sepsis.

Milk fat globule-EGF factor VIII (MFG-E8), also known as lactadherin, is a 66-kDa glycoprotein originally discovered in mouse milk and mammary epithelium (Stubbs et al, 1990). It is an important milk mucin-associated defense component that inhibits enterie pathogen binding and infectivity (Yolken, et al., 1992). MFG-E8 was subsequently found to be widely distributed in various tissues in mice and other mammalian species including humans (Aziz et al. 2009; Hanayama et al. 2004; Larocca et al. 1991). In the brain, MFG-E8 is expressed in astrocytes (Boddaert et al. 2007) and microglia (Fuller and Van Eldik 2008). MFG-E8 contains two N-terminals epidermal growth factor (EGF)-like repeats, and two C-terminal discoidin/F5/8C domains. MFG-E8 binds αᵥβ_{3/5} integrin heterodimers through an arginine-glycine-aspartic acid (RGD) motif contained in the second EGF domain (Anderson et al. 1997).

Recent studies have shown that MFG-E8 can also be secreted by activated macrophages and immature dendritic cells and has been linked to the opsonization of apoptotic cells (Hanayama, et al., 2002; Hanayama, et al., 2004; Miyasaka, et al., 2004; Thery, et al., 1999; Oshima, et al., 2002). The second F5/8C domain of MFG-E8 has high affinity for anionic membrane phospholipids such as phosphatidylserine that become externalized during apoptosis (Anderson et al, 1997; Shao et al. 2008). MFG-E8 has been shown to facilitate phagocytic removal of apoptotic cells by acting as a bridging molecule between phosphatidylserine exposed on the apoptotic cell and αᵥβ_{3/5} integrin receptors on phagocytes. This enhanced clearance of apoptotic cells prevents secondary necrosis which could release proinflammatory mediators leading to tissue damage (Hanayama et al. 2002), MFG-E8 also exerts other beneficial effects in tissue injury such as suppression of inflammation and apoptosis in intestinal ischemia (Cui et al. 2010) and Alzheimer's disease (Fuller and Van Eldik 2008).

Previous studies have shown that administration of rat MFG-E8-containing exosomes or recombinant murine MFG-E8 (rmMFG-E8) increases phagocytosis of apoptotic cells, reduces proinflammatory cytokines, and improves survival in a rat model of sepsis induced by cecal ligation of puncture (CLP) (Miksa, et al., 2008; Miksa, et al., 2009c). However, one obstacle hampering the development of MFG-E8 as a therapeutic agent for patients is the potential immunogenicity of animal proteins in humans.

The present invention addresses the need for treatment of cerebral ischemia and sepsis as well as other diseases and disorders, using in particular recombinant human MFG-E8 (rhMFG-E8).

### SUMMARY OF THE INVENTION

The present invention provides methods of preventing and/or treating cerebral ischemia in a subject comprising administering to the subject a milk fat globule epidermal growth factor-factor VIII (MFG-E8) in an amount effective to prevent and/or treat cerebral ischemia.

The invention also provides methods of preparing pharmaceutical compositions for preventing and/or treating cerebral ischemia, the methods comprising formulating milk fat globule epidermal growth factor-factor VIII (MFG-E8) in a pharmaceutical composition in an amount effective to prevent and/or treat cerebral ischemia.

The invention also provides pharmaceutical compositions comprising milk fat globule epidermal growth factor-factor VIII (MFG-E8) in dosage form for preventing and/or treating cerebral ischemia, and a pharmaceutically acceptable carrier.

The invention further provides pharmaceutical products comprising a milk fat globule epidermal growth factor-factor VIII (MFG-E8) formulated in a pharmaceutically acceptable carrier; and a package insert providing instructions for the administration of MFG-E8 for the prevention and/or treatment of cerebral ischemia.

The invention also provides recombinant human MFG-E8 (rhMFG-E8) having an amino acid sequence that is at least 95% identical to human MFG-E8 (hMFG-E8) (SEQ ID NO:1), wherein the rhMFG-E8 is non-glycosylated.

The invention also provides methods of preventing and/or treating inflammation and/or organ injury after ischemia/reperfusion in a subject comprising administering to the subject a recombinant human milk fat globule epidermal growth factor-factor VIII (rhMFG-E8) in an amount effective to prevent and/or treat inflammation and/or organ injury, wherein the rhMFG-E8 has an amino acid sequence that is at least 95% identical to human MFG-E8 (hMFG-E8) (SEQ ID NO:1) and wherein the rhMFG-E8 is non-glycosylated.

The invention further provides methods of treating a subject having sepsis or a subject at risk for sepsis, the methods comprising administering to the subject an amount of a recombinant human milk fat globule epidermal growth factor-factor VIII (rhMFG-E8) effective to reduce a physiologic effect of sepsis, wherein the rhMFG-E8 has an amino acid sequence that is at least 95% identical to human MFG-E8 (hMFG-E8) (SEQ ID NO:1) and wherein the rhMFG-E8 is non-glycosylated.

The invention also provides methods of treating lung injury in a subject comprising administering to the subject an amount of a recombinant human milk fat globule epidermal growth factor-factor VIII (rhMFG-E8) effective to treat lung injury, wherein the rhMFG-E8 has an amino acid sequence that is at least 95% identical to human MFG-E8 (hMFG-E8) (SEQ ID NO:1) and wherein the rhMFG-E8 is non-glycosylated.

Also provided are methods of preparing a pharmaceutical composition for preventing and/or treating inflammation and/or organ injury after ischemia/reperfusion in a subject, the methods comprising formulating a recombinant human milk fat globule epidermal growth factor-factor VIII (rhMFG-E8) in a pharmaceutical composition in an amount effective to prevent and/or treat inflammation and/or organ injury after ischemia/reperfusion, wherein the rhMFG-E8 has an amino acid sequence that is at least 95% identical to human MFG-E8 (hMFG-E8) (SEQ ID NO:1) and wherein the rhMFG-E8 is non-glycosylated.

Also provided are methods of preparing a pharmaceutical composition for treating a subject having sepsis or a subject at risk for sepsis, the methods comprising formulating a recombinant human milk fat globule epidermal growth factor-factor VIII (rhMFG-E8) in a pharmaceutical composition in an amount effective to reduce a physiologic effect of sepsis, wherein the rhMFG-E8 has an amino acid sequence that is at least 95% identical to human MFG-E8 (hMFG-E8) (SEQ ID NO:1) and wherein the rhMFG-E8 is non-glycosylated.

Further provided are methods of preparing a pharmaceutical composition for treating lung injury in a subject, the methods comprising formulating a recombinant human milk fat globule epidermal growth factor-factor VIII (rhMFG-E8) in a pharmaceutical composition in an amount effective to treat lung injury, wherein the rhMFG-E8 has an amino acid sequence that is at least 95% identical to human MFG-E8 (hMFG-E8) (SEQ ID NO:1) and wherein the rhMFG-E8 is non-glycosylated.

Also provided are pharmaceutical compositions comprising a recombinant human milk fat globule epidermal growth factor-factor VIII (rhMFG-E8) in dosage form for preventing and/or treating inflammation and/or organ injury after ischemia/reperfusion, and a pharmaceutically acceptable carrier, wherein the rhMFG-E8 has an amino acid sequence that is at least 95% identical to human MFG-E8 (hMFG-E8) (SEQ ID NO:1) and wherein the rhMFG-E8 is non-glycosylated.

Also provided are pharmaceutical compositions comprising a recombinant human milk fat globule epidermal growth factor-factor VIII (rhMFG-E8) in dosage form for treating a subject having sepsis or a subject at risk for sepsis, and a pharmaceutically acceptable carrier, wherein the rhMFG-E8 has an amino acid sequence that is at least 95% identical to human MFG-E8 (hMFG-E8) (SEQ ID NO:1) and wherein the rhMFG-E8 is non-glycosylated.

Further provided are pharmaceutical compositions comprising a recombinant human milk fat globule epidermal growth factor-factor VIII (rhMFG-E8) in dosage form for treating lung injury, and a pharmaceutically acceptable carrier, wherein the rhMFG-E8 has an amino acid sequence that is at least 95% identical to human MFG-E8 (hMFG-E8) (SEQ ID NO:1) and wherein the rhMFG-E8 is non-glycosylated.

Also provided are pharmaceutical products comprising a recombinant human milk fat globule epidermal growth factor-factor VIII (rhMFG-E8) formulated in a pharmaceutically acceptable carrier; and a package insert providing instructions for the administration of rhMFG-E8 for the prevention and/or treatment of inflammation and/or organ injury after ischemia/reperfusion, wherein the rhMFG-E8 has an amino acid sequence that is at least 95% identical to human MFG-E8 (hMFG-E8) (SEQ ID NO:1) and wherein the rhMFG-E8 is non-glycosylated.

Also provided are pharmaceutical products comprising a recombinant human milk fat globule epidermal growth factor-factor VIII (rhMFG-E8) formulated in a pharmaceutically acceptable carrier; and a package insert providing instructions for the administration of rhMFG-E8 for treating a subject having sepsis or a subject at risk for sepsis, wherein the rhMFG-E8 has an amino acid sequence that is at least 95% identical to human MFG-E8 (hMFG-E8) (SEQ ID NO:1) and wherein the rhMFG-E8 is non-glycosylated.

Further provided are pharmaceutical products comprising a recombinant human milk fat globule epidermal growth factor-factor VIII (rhMFG-E8) formulated in a pharmaceutically acceptable carrier; and a package insert providing instructions for the administration of rhMFG-E8 for the treatment of lung injury, wherein the rhMFG-E8 has an amino acid sequence that is at least 95% identical to human MFG-E8 (hMFG-E8) (SEQ ID NO:1) and wherein the rhMFG-E8 is non-glycosylated.

### BRIEF DESCRIPTION OF THE DRAWINGS

FIG. 1. SDS-PAGE analysis of the expressed and purified rhMFG-E8 ; Lane 1, purified rhMFG-E8 (1 μg); Lane 2; purified rhMFG-E8 (0.5 μg); Lane 3, marker; Lane 4, unpurified bacterial lysis.

FIG. 2. Western blot analysis of the expressed and purified rhMFG-E8. The specific anti-human antibody recognizes MFG-E8 by western blot analysis. Lane 1, purified rhMFG-E8 (1 μg); lane 2, marker.

FIG. 3. rhMFG-E8 potentiates phagocytosis of apoptotic cells. Fluorescent intensity of pHrodo-SE-labled apoptotic thymocytes increases after engulfment by macrophages. Splenic macrophages were labeled with FITC anti-CD11b/c and thymocytes with pHrodo-SE. Cells were co-incubated for 60 min, collected and fixed with 1% PFA prior to fluorescent microscopy. Phagocytosis index of CD11b/c+pHrodo+ cells is shown in the figure. Data are presented as means±SE (n=6-9/group) and compared by one-way ANOVA and Student-Newman-Keuls Method: *P<0.05 vs. medium alone.

FIG. 4a-4b. rhMFG-E8 reduced thymocyte apoptosis after CLP. Rats underwent CLP to induce experimental sepsis and were treated with human albumin (Vehicle), rmMFG-E8 (20 μg/kg BW), or rhMFG-E8 (20 μg/kg BW) immediately after CLP. (A) Thymocyte apoptosis was assessed 20 h after CLP by Annexin V/PI staining and FACS analysis. Data are presented as means±SE (n=4/group) and compared by one-way ANOVA and Student-Newman-Keuls Method: *P<0.05 vs. Sham group; #P<0.05 vs. Vehicle group. (B) Alterations in cleaved caspase-3 in the thymus were determined by Western Blot at 20 h after CLP. Representative gels of 2 independent observations are presented.

FIG. 5a-5b. rhMFG-E8 attenuated organ injury after CLP. Rats underwent CLP to induce experimental sepsis and were treated with human albumin (Vehicle), rmMFG-E8 (20 μg/kg BW), or rhMFG-E8 (20 μg/kg BW) immediately after CLP. Serum levels of lactate (A) and IL-6 (B) were measured at 20 h after CLP. Data are presented as means±SE (n=4-6/group) and compared by one-way ANOVA and Student-Newman-Keuls Method: *P<0.05 vs. Sham group; #P<0.05 vs. Vehicle group.

FIG. 6. Treatment with rmMFG-E8 or rhMFG-E8 improves survival rate at 10 days after cecal ligation and puncture. After CLP, mice were given human albumin treatment (Vehicle) or rmMFG-E8 (20 μg/kg BW), or rhMFG-E8 (20 μg/kg BW) treatment. There were 20 animals in each group. The survival rates were estimated by the Kaplan-Meier method and compared by using the log-rank test. *P<0.05 vs. vehicle group.

FIG. 7. Alteration in cerebral MFG-E8 level after focal cerebral ischemia by middle cerebral artery occlusion (MCAO). Cerebral MFG-E8 levels were measured at 24 h post-MCAO. Data are presented as mean±SE, and analyzed by Student's t-test. Compared with Sham, cerebral ischemia (MCAO) decreased MFG-E8 levels (n=4-5, *p<0.05 vs. Sham).

FIG. 8. rhMFG-E8 treatment decreases neurological deficit after focal cerebral ischemia. A combined neurological deficit score (combined neuroscore) was determined by assessing sensorimotor and reflex behavioral deficits at 24 h after cerebral ischemia. Data are presented as mean±SE, and analyzed by one-way ANOVA and Student Newman Keul's method. Compared with Sham, cerebral ischemia caused significant neurological deficits in both vehicle and rhMFG-E8 treated animals. rhMFG-E8 treatment significantly reduced neurological deficit compared with vehicle group (n=6, *p<0.05 vs. Sham, #p<0.05 vs. Vehicle).

FIG. 9a-9c. Alterations in infarct size and cerebral histopathology in sham-operated rats (Sham) compared with vehicle and rhMFG-E8 treated after cerebral ischemia. (a) 2 mm thick coronal slices of fresh brain tissue were stained with triphenyl tetrazolium chloride (ITC) and digitally analyzed with NIH ImageJ software. Representative images of TTC staining are shown at the top of the bars. Data are presented as mean±SE, and analyzed by Student's t-test. Treatment with rhMFG-E8 decreased infarct size compared with Vehicle (n=6, *p<0.05 vs. Vehicle). (b) Photomicrographs of hematoxylin-eosin (H & E) stained slides were obtained by scanning slides (A, C, E), and by bright field microscopy at 400× original magnification (B, D, F). Sham (A, B) shows only normal viable basophilic neurons (shown by arrow heads). Vehicle group (C, D) shows mainly necrotic eosinophilic neurons (shown by long arrows), rhMFG-E8 treatment (E, F) protects against neuronal necrosis resulting in a mix of viable basophilic neurons and necrotic eosinophilic neurons. Scale bar=50 μm. (c) The average number of intact neurons (basophilic neurons) in six random fields per H & E stained slide was determined. Data are presented as mean±SE, and analyzed by Kruskal-Wallis one-way ANOVA. Cerebral ischemia (MCAO) caused significant reduction in number of intact neurons in both Vehicle and rhMFG-E8 treated animals compared with Sham animals. rhMFG-E8 treatment protected neurons against necrosis (n=6, *p<0.05 vs. Sham, #p<0.05 vs. Vehicle).

FIG. 10a-10d. Alterations in cerebral IL-6, TNFα, and myeloperoxidase in sham-operated rats (Sham) compared with vehicle and rhMFG-E8 treatment after cerebral ischemia. (a) Cerebral IL-6 levels were measured by ELISA at 24 h post-MCAO. Data are presented as mean±SE, and analyzed by one-way ANOVA and Student Newman Keul's method. Cerebral ischemia (MCAO) caused elevation of IL-6 levels in both Vehicle and rhMFGE8-treated animals compared with Sham animals. Treatment with rhMFG-E8 downregulated IL-6 expression compared with Vehicle (n=6, *p<0.05 vs. Sham, #p<0.05 vs. Vehicle). (b) Brain tissue was immunohistochemically stained for TNF-α and examined at 400× original magnification under bright field microscopy. Vehicle (B) and rhMFG-E8 (C) treated animals showed increased expression of TNF-α compared with Sham animals (A). rhMFG-E8 treatment (C) decreased TNF-α expression compared with Vehicle (B). Scale bar=50 μm. (c) Brain issue immunohistochemically stained for neutrophil marker, myeloperoxidase, and examined under bright field microscopy at 400× original magnification. Sham (A) animals showed no staining for myeloperoxidase (no neutrophil infiltration) whereas Vehicle (B) and rhMFG-E8 (C) treated animals showed staining for myeloperoxidase. rhMFG-E8 treatment (C) after cerebra ischemia decreased myeloperoxidase staining compared with Vehicle (B). Scale bar=50 μm. (d) Quantification of cerebral neutrophil infiltration by myeloperoxidase immunohistochemistry. Neutrophils were identified as small, round, myeloperoxidase-staining cells on bright field microscopy at 400× original magnification. The average number of neutrophils in six random fields per slide was determined as neutrophil count/40× high power field (hpf). Data are presented as mean±SE, and analyzed by oneway ANOVA and Student Newman Keul's method. Compared with Sham animals, Vehicle arid rhMFG-E8 treated animals showed increased neutrophil infiltration. rhMFGE8 treatment decreased cerebral neutrophil infiltration compared with vehicle (n=4, *p<0.05 vs. Sham, #p<0.05 vs. Vehicle).

FIG. 11a-11b. Alteration in ICAM-1 and peroxisome proliferator activated receptor-γ (PPAR-γ) expression after cerebral ischemia. (a) Cerebral ICAM-1 gene expression was measured by RT-PCR. Data are presented as mean±SE, and analyzed by one-way ANOVA and Student Newman Keul's method. Cerebral ischemia resulted in upregulation of ICAM-1 expression in Vehicle compared with Sham. rhMFG-E8 treatment decreased ICAM-1 expression, even though not significant compared with Vehicle (n=4-6, *p<0.05 vs. Sham). (b) PPAR-γ protein levels were determined by western blot at 24 h post-MCAO. Data are presented as mean±SE, and analyzed by Kruskal-Wallis one-way ANOVA. Compared with Sham, cerebral ischemia downregulated PPAR-γ in the Vehicle group. rhMFG-E8 treatment upregulated PPAR-γ expression compared with Vehicle (n=6, *p<0.05 vs. Sham, #p<0.05 vs. Vehicle).

FIG. 12a-12c. The effects of rhMFG-E8 treatment on apoptosis, measured by Bcl-2/Bax ratio and TUNEL staining, after cerebral ischemia in rats. (a) Bcl-2/Bax ratio was determined by western blot. Data are presented as means±SE, and analyzed by one-way ANOVA and Student Newman Keul's method. The Bcl-2/Bax ratio was not different between Vehicle and Sham. rhMFG-E8 treatment significantly elevated Bcl-2/Bax ratio compared with Vehicle and Sham (n=6, *p<0.05 vs. Vehicle and Sham), (b) TUNEL staining after cerebral ischemia. On fluorescent microscopy at 200× original magnification, apoptotic cells appeared as brighter fluorescent while propidium iodide (PI) darker staining showed the nuclear location of the TUNEL reaction products. The Sham group (A, B, C) showed no apoptosis since there were no positive cells on TUNEL staining (A). The Vehicle group (D, E, F) shows increased apoptosis as shown by increased number of TUNEL positive cells (D). A merge (F) of TUNEL staining (D) and PI staining (E) shows that most of the cells in the penumbra of Vehicle animals were apoptotic. Treatment with rhMFG-E8 (G, H, I) decreased apoptosis as shown by less TUNEL staining (G) compared with Vehicle TUNEL staining (D). A merge (I) of the rhMFG-E8 TUNEL staining (G) and PI staining (H) shows that rhMFG-E8 treatment protected brain cells from apoptosis compared with the Vehicle group (F). (c) Quantification of TUNEL staining. Eight random fields were captured at 200× original magnification for each slide. The average number of TUNEL positive cells were counted and expressed as TUNEL cells/20× high power field (hpf). Data are presented as mean±SE, and analyzed by oneway ANOVA and Student Newman Keul's method. Cerebral ischemia increased TUNEL-positive cells in Vehicle and rhMFG-E8 treated animals compared with Sham animals. Treatment with rhMFG-E8 decrease the number of TUNEL-positive cells compared with Vehicle group (n=4, *p<0.05, Sham, #p<0.05 vs. Vehicle).

### DETAILED DESCRIPTION OF THE INVENTION

The present invention provides methods of preventing and/or treating cerebral ischemia in a subject comprising administering to the subject a milk fat globule epidermal growth factor-factor VIII (MFG-E8) in an amount effective to prevent and/or treat cerebral ischemia.

The subject can be, for example, a subject having cerebral ischemia or a patient at risk for cerebral ischemia, for example, a patient who is undergoing or about to undergo surgery. The cerebral ischemia can be. for example, a focal brain ischemia caused by a blood clot that occludes a cerebral blood vessel, or global brain ischemia caused by reduced blood flow to the brain.

As used herein, to “treat” cerebral ischemia in a subject means to prevent or reduce a physiological effect of cerebral ischemia. For example, administration of MFG-E8 to the subject can reduce cerebral level of interleukin-6 (IL-6), and/or reduce numbers of infiltrated neutrophils, and/or reduce cerebral inflammation and/or apoptosis. Preferably, administration of MFG-E8 reduces and/or prevents death of brain tissue. Preferably, the chance of survival of the subject is increased by the administration of MFG-E8.

The invention also provides a method of preparing a pharmaceutical composition for preventing and/or treating cerebral ischemia, the method comprising formulating milk fat globule epidermal growth factor-factor VIII (MFG-E8) in a pharmaceutical composition in an amount effective to prevent and/or treat cerebral ischemia.

The invention also provides a pharmaceutical composition comprising milk fat globule epidermal growth factor-factor VIII (MFG-E8) in dosage form for preventing and/or treating cerebral ischemia, and a pharmaceutically acceptable carrier.

The invention further provides a pharmaceutical product comprising a milk fat globule epidermal growth factor-factor VIII (MFG-E8) formulated in a pharmaceutically acceptable carrier; and a package insert providing instructions for the administration of MFG-E8 for the prevention and/or treatment of cerebral ischemia.

In a preferred embodiment of any of the methods, compositions, products or uses described herein, the MFG-E8 is a recombinant human MFG-E8 (rhMFG-E8). In different embodiments, the rhMFG-E8 has an amino acid sequence that is at least 95% identical to human MFG-E8 (hMFG-E8) (SEQ ID NO:1), or that is at least 99% identical to human MFG-E8 (hMFG-E8) (SEQ ID NO:1), or that is identical to human MFG-E8 (hMFG-E8) (SEQ ID NO:1). In a preferred embodiment, the MFG-E8 is non-glycosylated.

The invention also provides a recombinant human MFG-E8 (rhMFG-E8) having an amino acid sequence that is a least 95% identical to human MFG-E8 (hMFG-E8) (SEQ ID NO:1), wherein the rhMFG-E8 is non-glycosylated.

The invention also provides a method of preventing and/or treating inflammation and/or organ injury after ischemia/reperfusion in a subject comprising administering to the subject a recombinant human milk fat globule epidermal growth factor-factor VIII (rhMFG-E8) in an amount effective to prevent and/or treat inflammation and/or organ injury, wherein the rhMFG-E8 has an amino acid sequence that is at least 95% identical to human MFG-E8 (hMFG-E8) (SEQ ID NO:1) and wherein the rhMFG-E8 is non-glycosylated. The ischemia/reperfusion can be, the example, one or more of gastrointestinal tract, liver, lung, kidney, heart, brain, spinal cord or crushed limb ischemia/reperfusion.

Preferably, the method prevents or reduces serum elevation of one or more of tumor necrosis factor-α, interleukin-6, interleukin-1β, aspartate aminotransferase, alanine aminotransferase, lactate, or lactate dehydrogenase. Preferably, inflammation is prevented or treated. Preferably, organ injury is prevented or treated, where for example, the organ is one or more of gastrointestinal tract, liver, lung, kidney, heart, brain, spinal cord or crushed limb. Preferably, the chance of survival of the subject is increased.

The invention further provides a method of treating a subject having sepsis or a subject at risk for sepsis, the method comprising administering to the subject an amount of a recombinant human milk fat globule epidermal growth factor-factor VIII (rhMFG-E8) effective to reduce a physiologic effect of sepsis, wherein the rhMFG-E8 has an amino acid sequence that is at least 95% identical to human MFG-E8 (hMFG-E8) (SEQ ID NO:1) and wherein the rhMFG-E8 is non-glycosylated. The physiologic effect of sepsis can be, for example, elevation of serum TNF-α levels, and/or elevation of serum IL-6 levels, and/or shock. Preferably, administration of rhMFG-E8 attenuates systemic inflammation.

The invention also provides a method of treating lung injury in a subject comprising administering to the subject an amount of a recombinant human milk fat globule epidermal growth factor-factor VIII (rhMFG-E8) effective to treat lung injury, wherein the rhMFG-E8 has an amino acid sequence that is an least 95% identical in human MFG-E8 (hMFG-E8) (SEQ ID NO:1) and wherein the rhMFG-E8 is non-glycosylated. In a preferred embodiment, the lung injury is an acute lung injury.

The use of forms of MFG-E8 other than the rhMFG-E8 disclosed herein for treating sepsis, ischemia/reperfusion and lung injury have been described (US 2009/0297498, WO 2009/064448).

The invention also provides a method of preparing a pharmaceutical composition for preventing and/or treating inflammation and/or organ injury after ischemia/reperfusion in a subject, the method comprising formulating a recombinant human milk fat globule epidermal growth factor-factor VIII (rhMFG-E8) in a pharmaceutical composition in an amount effective to prevent and/or treat inflammation and/or organ injury after ischemia/reperfusion, wherein the rhMFG-E8 has an amino acid sequence that is at least 95% identical to human MFG-E8 (hMFG-E8) (SEQ ID NO:1) and wherein the rhMFG-E8 is non-glycosylated.

The invention also provides a method of preparing a pharmaceutical composition for treating a subject having sepsis or a subject at risk for sepsis, the method comprising formulating a recombinant human milk fat globule epidermal growth factor-factor VIII (rhMFG-E8) in a pharmaceutical composition in an amount effective to reduce a physiologic effect of sepsis, wherein the rhMFG-E8 has an amino acid sequence that is at least 95% identical to human MFG-E8 (hMFG-E8) (SEQ ID NO:1) and wherein the rhMFG-E8 is non-glycosylated.

The invention further provides a method of preparing a pharmaceutical composition for treating lung injury in a subject, the method comprising formulating a recombinant human milk fat globule epidermal growth factor-factor VIII (rhMFG-E8) in a pharmaceutical composition in an amount effective to treat lung injury, wherein the rhMFG-E8 has an amino acid sequence that is at least 95% identical to human MFG-E8 (hMFG-E8) (SEQ ID NO:1) and wherein the rhMFG-E8 is non-glycosylated.

The invention also provides a pharmaceutical composition comprising a recombinant human milk fat globule epidermal growth factor-factor VIII (rhMFG-E8) in dosage form for preventing and/or treating inflammation and/or organ injury after ischemia/reperfusion, and a pharmaceutically acceptable carrier, wherein the rhMFG-E8 has an amino acid sequence that is at least 95% identical to human MFG-E8 (hMFG-E8) (SEQ ID NO:1) and wherein the rhMFG-E8 is non-glycosylated.

The invention also provides a pharmaceutical composition comprising a recombinant human milk fat globule epidermal growth factor-factor VIII (rhMFG-E8) in dosage form for treating a subject having sepsis or a subject at risk for sepsis, and a pharmaceutically acceptable carrier, wherein the rhMFG-E8 has an amino acid sequence that is at least 95% identical to human MFG-E8 (hMFG-E8) (SEQ ID NO:1) and wherein the rhMFG-E8 is non-glycosylated.

The invention further provides a pharmaceutical composition comprising a recombinant human milk fat globule epidermal growth factor-factor VIII (rhMFG-E8) in dosage form for treating lung injury, and a pharmaceutically acceptable carrier, wherein the rhMFG-E8 has an amino acid sequence that is a least 95% identical to human MFG-E8 (hMFG-E8) (SEQ ID NO:1) and wherein the rhMFG-E8 is non-glycosylated.

The invention also provides a pharmaceutical product comprising a recombinant human milk fat globule epidermal growth factor-factor VIII (rhMFG-E8) formulated in a pharmaceutically acceptable carrier; and a package insert providing instructions for the administration of rhMFG-E8 for the prevention and/or treatment of inflammation and/or organ injury after ischemia/reperfusion, wherein the rhMFG-E8 has an amino acid sequence that is at least 95% identical to human MFG-E8 (hMFG-E8) (SEQ ID NO:1) and wherein the rhMFG-E8 is non-glycosylated.

The invention also provides a pharmaceutical product comprising a recombinant human milk fat globule epidermal growth factor-factor VIII (rhMFG-E8) formulated in a pharmaceutically acceptable carrier; and a package insert providing instructions for the administration of rhMFG-E8 for treating a subject having sepsis or a subject at risk for sepsis, wherein the rhMFG-E8 has an amino acid sequence that is at least 95% identical to human MFG-E8 (hMFG-E8) (SEQ ID NO:1) and wherein the rhMFG-E8 is non-glycosylated.

The invention further provides a pharmaceutical product comprising a recombinant human milk fat globule epidermal growth factor-factor VIII (rhMFG-E8) formulated in a pharmaceutically acceptable carrier; and a package insert providing instructions for the administration of rhMFG-E8 for the treatment of lung injury, wherein the rhMFG-E8 has an amino acid sequence that is at least 95% identical to human MFG-E8 (hMFG-E8) (SEQ ID NO:1) and wherein the rhMFG-E8 is non-glycosylated.

The invention also provides for the use of a milk fat globule epidermal growth factor-factor VIII (MFG-E8) for the preparation of a medicament for the prevention and/or treatment of cerebral ischemia, as well as a milk fat globule epidermal growth factor-factor VIII (MFG-E8) for use for preventing and/or treating cerebral ischemia.

The invention further provides for the use of a recombinant human milk fat globule epidermal growth factor-factor VIII (rhMFG-E8) for the preparation of a medicament for the prevention and/or treatment of inflammation and/or organ injury after ischemia/reperfusion, or for the treatment of a subject having sepsis or a subject at risk for sepsis, or for the treatment of lung injury, wherein the rhMFG-E8 has an amino acid sequence that is at least 95% identical to human MFG-E8 (hMFG-E8) (SEQ ID NO:1) and wherein the rhMFG-E8 is non-glycosylated, as well providing rhMFG-E8 for use for preventing and/or treating inflammation and/or organ injury after ischemia/reperfusion, or for treating a subject having sepsis or a subject at risk for sepsis, or for treating lung injury in a subject.

In different embodiments of the recombinant human MFG-E8, methods, compositions, products, or uses the rhMFG-E8 has an amino acid sequence that is at least 99% identical to human MFG-E8 (hMFG-E8) (SEQ ID NO:1) or the rhMFG-E8 has an amino acid sequence that is identical to human MFG-E8 (hMFG-E8) (SEQ ID NO:1).

MFG-E8 can be administered to the subject in a pharmaceutical composition comprising a pharmaceutically acceptable carrier. Examples of acceptable pharmaceutical carriers include, but are not limited to, additive solution-3 (AS-3), saline, phosphate buffered saline, Ringer's solution, lactated Ringer's solution, Locke-Ringer's solution, Krebs Ringer's solution, Hartmann's balanced saline solution, and heparinized sodium citrate acid dextrose solution.

Compositions comprising MFG-E8 can be formulated without undue experimentation for administration to a subject, including humans, as appropriate for the particular application. Additionally, proper dosages of the compositions can be determined without undue experimentation using standard dose-response protocols.

Accordingly, the compositions designed for oral, lingual, sublingual, buccal and intrabuccal administration can be made without undue experimentation by means well known in the art, for example with an inert diluent or with an edible carrier. The compositions may be enclosed in gelatin capsules or compressed into tablets. For the purpose of oral therapeutic administration, the pharmaceutical compositions of the present invention may be incorporated with excipients and used in the form of tablets, troches, capsules, elixirs, suspensions, syrups, wafers, chewing gums and the like.

Tablets, pills, capsules, troches and the like may also contain binders, recipients, disintegrating agent, lubricants, sweetening agents, and flavoring agents. Some examples of binders include microcrystalline, cellulose, gum tragacanth or gelatin. Examples of excipients include starch or lactose. Some examples of disintegrating agents include alginic acid, corn starch and the like. Examples of lubricants include magnesium stearate or potassium stearate. An example of a glidant is colloidal silicon dioxide. Some examples of sweetening agents include sucrose, saccharin and the like. Examples of flavoring agents include peppermint, methyl salicylate, orange flavoring and the like. Materials used in preparing these various compositions should be pharmaceutically pure and nontoxic in the amounts used.

The compositions of the present invention can easily be administered parenterally such as for example, by intravenous, intramuscular, intrathecal or subcutaneous injection. Parenteral administration can be accomplished by incorporating the compositions of the present invention into a solution or suspension. Such solutions or suspensions may also include sterile diluents such as water for injection, saline solution, fixed oils, polyethylene glycols, glycerine, propylene glycol or other synthetic solvents. Parenteral formulations may also include antibacterial agents such as for example, benzyl alcohol or methyl parabens, antioxidants such as for example, ascorbic acid or sodium bisulfite and chelating agents such as EDTA. Buffers such as acetates, citrates or phosphates and agents for the adjustment of tonicity such as sodium chloride or dextrose may also be added. The parenteral preparation can be enclosed in ampules, disposable syringes or multiple dose vials made of glass or plastic.

Rectal administration includes administering the pharmaceutical compositions into the rectum or large intestine. This can be accomplished using suppositories or enemas. Suppository formulations can easily be made by methods known in the art. For example, suppository formulations can be prepared by heating glycerin to about 120° C., dissolving the composition in the glycerin, mixing the heated glycerin after which purified water may be added, and pouring the hot mixture into a suppository mold.

Transdermal administration includes percutaneous absorption of the composition through the skin. Transdermal formulations include patches (such as the well-known nicotine patch), ointments, creams, gels, salves and the like.

The present invention includes nasally administering to the mammal a therapeutically effective amount of the composition. As used herein, nasally administering or nasal administration includes administering the composition to the mucous membranes of the nasal passage or nasal cavity of the patient. As used herein, pharmaceutical compositions for nasal administration of a composition include therapeutically effective amounts of the composition prepared by well-known methods to be administered, for example, as a nasal spray, nasal drop, suspension, gel, ointment, cream or powder. Administration of the composition may also take place using a nasal tampon or nasal sponge.

The subject can be a human or another animal.

Amino acid sequences for human and mouse MFG-E8 are shown below.

| SEQ ID NO: 2 | |
|---|---|
| human MFG-E8 - from GenBank NP005919 | |
| 1 | mprprliaal egallcapsl lvaldicskn pchngglcee isqevrgdvf psytctclkg |
| 61 | yagnheetke veplgmengn iansqiaass vrvtflglqh wvpelarlnr agmvnawtps |
| 121 | snddnpwiqv nllrrmwvtg vvtqgasrla sheylkafkv ayslnghefd fihdvnkkhk |
| 181 | efvgnwnkna vhvnlfetpv eaqyvrlypt schtactlrf ellgcelngc anplglknns |
| 241 | ipdkqitass syktwglhlf swnpsyarld kqgnfnawva gsygndqwlq vdlgsskevt |
| 301 | giitqgarnf gsvqfvasyk vaysndsanw teyqdprtgs skifpgnwdn hshkknlfet |
| 361 | pilaryvril pvawhnrial rlellgc |

Human MGF-E8 protein is synthesized as the 387 amino acid precursor shown above that contains a 23 amino acid signal sequence and a 364 amino acid mature region. The recombinant human protein expressed in this study is the mature molecule of human MFG-E8 (i.e., Leu24-Cys387), i.e., amino acids 24 through 387 of SEQ ID NO:2, which is herein referred to a SEQ ID NO:1.

| SEQ ID NO: 3 | |
|---|---|
| mouse MFG-E8 - from GenBank NP032620 | |
| 1 | mqvsrvlaal cgmllcasgl faasgdfcds slclnggtcl tgqdndiycl cpegftglve |
| 61 | netergpcsp npcyndakcl vtldtqrgdi fteyicqcpv gysgihcete tnyynldgey |
| 121 | mfttavpnta vptpaptpdl snnlasrcst qlgmeggaia dsqisasyvy mgfmglqrwg |
| 181 | pelarlyrtg ivnawhasny dskpwiqvnl lrkmrvsgvm tqgasragra eylktfkvay |
| 241 | sldgrkfefi qdesggdkef lgnldnnslk vnmfnptlea qyirlypvsc hrgctlrfel |
| 301 | lgcelhgcle plglknntip dspmsasssy ktwnlrafgw yphlgrldnq gkinawtaqs |
| 361 | nsakewlqvd lgtqrqvtgi itqgardfgh iqyvesykva hsddgvqwtv yeeqgsskvf |
| 421 | qgnldnnshk knifekpfms ryvrvlpvsw hnritlrlel lgc |

This invention will be better understood from the Experimental Details, which follow. However, one skilled in the art will readily appreciate that the specific methods and results discussed are merely illustrative of the invention as described more fully in the claims that follow thereafter.

### EXPERIMENTAL DETAILS

### Example I

### Recombinant Human MFG-E8 and Treatment of Sepsis

### Materials and Methods

### Expression of Recombinant Human MFG-E8

A 1095 bp fragment (SEQ ID NO:3) encoding the mature region of human MFG-E8 (364 amino acids, R24-R387, SwissProt #: Q08431) was obtained by polymerase chain reaction amplification of a plasmid template containing the human MFG-E8 cDNA. The open reading frame was cloned into the Sal I and Not I site of the pET-28a(+) vector (Novagen, Madison, Wis.) downstream of the phage T7 RNA polymerase promoter. The final protein product contains six histidines fused to the N-terminus of human MFG-E8. The plasmid was transformed into E. coli BL21 (DE3) cells. The cells were grown at 37° C. in 2YT medium (Invitrogen) with kanamycin overnight. The rhMFG-E8 protein production was induced by the addition of isopropyl-β-D-thiogalactopyranoside (IPTG) to a final concentration of 1.0 mM and cells growth continued for 5 h at 25° C. The cells were harvested by centrifugation and the induced rhMFG-E8 protein was purified according to the manufacture's instruction (Novagen). The rhMFG-E8 fractions were pooled and endotoxin of protein solution was removed by phase separation using Triton X-114 (Aida and Pabst. 1990). The content of LPS in the sample was determined using the Limulus amebocyte lysate assay (BioWhittaker Inc, Walkersville, Md.) are described previously (Li, et al., 2004). The purity of rhMFG-E8 was evaluated by SDS-PAGE on a 10-20% Tris-HCl gel and visualized using GelCode Blue Stain Regent (Pierce, Rockford Ill.). The final product was concentrated by Amicon Ultra-15 Centrifugal Filter Devices to designed concentration and stored at −20° C. Human MFG-E8 encoding DNA sequence minus the signal peptide (SEQ ID NO:4):

| | |
|---|---|
| 1 | ctggatatct gttccaaaaa cccctgccac aacggtggtt tatgcgagga |
| 51 | gatttcccaa gaagtgcgag gagatgtctt cccctcgtac acctgcacgt |
| 101 | gccttaaggg ctacgcgggc aaccactgtg agacgaaatg tgtcgagcca |
| 151 | ctgggcatgg agaatgggaa cattgccaac tcacagatcg ccgcctcatc |
| 201 | tgtgcgtgtg accttcttgg gtttgcagca ttgggtcccg gagctggccc |
| 251 | gcctgaaccg cgcaggcatg gtcaatgcct ggacacccag cagcaatgac |
| 301 | gataacccct ggatccaggt gaacctgctg cggaggatgt gggtaacagg |
| 351 | tgtggtgacg cagggtgcca gccgcttggc cagtcatgag tacctgaagg |
| 401 | ccttcaaggt ggcctacagc cttaatggac acgaattcga tttcatccat |
| 451 | gatgttaata aaaaacacaa ggagtttgtg ggtaactgga acaaaaacgc |
| 501 | ggtgcatgtc aacctgtttg agacccctgt ggaggctcag tacgtgagat |
| 551 | tgtaccccac gagctgccac acggcctgca ctctgcgctt tgagctactg |
| 601 | ggctgtgagc tgaacggatg cgccaatccc ctgggcctga agaataacag |
| 651 | catccctgac aagcagatca cggcctccag cagctacaag acctggggct |
| 701 | tgcatctctt cagctggaac ccctcctatg cacggctgga caagcagggc |
| 751 | aacttcaacg cctgggttgc ggggagctac ggtaacgatc agtggctgca |
| 801 | ggtggacctg ggctcctcga aggaggtgac aggcatcatc acccaggggg |
| 851 | cccgtaactt tggctctgtc cagtttgtgg catcctacaa ggttgcctac |
| 901 | agtaatgaca gtgcgaactg gactgagtac caggacccca ggactggcag |
| 951 | cagtaagatc ttccctggca actgggacaa ccactcccac aagaagaact |
| 1001 | tgtttgagac gcccatcctg gctcgctatg tgcgcatcct gcctgtagcc |
| 1051 | tggcacaacc gcatcgccct gcgcctggag ctgctgggct gttag |

### Mass Spectrometry

The amino acid sequence of the isolated and purified protein was analyzed by LC-MS/MS at the Proteomics Resource Center of the Rockefeller University (New York, N.Y.). Briefly, the sample was reduced with 5 mM of DTT and alkylated with 10 mM iodoacetamide, and then digested with Sequence Grade Modified Trypsin (Promega) in ammonium bicarbonate buffer at 37° C. overnight. The digestion products were analyzed by LC-MS/MS. For LC-MS/MS analysis, the digestion product was separated by gradient elution with the Dionex capillary/nano-HPLC system and analyzed by Applied Biosystems QSTAR XL mass spectrometer using information-dependent, automated acquisition. The acquired ms/ms spectra were converted to a MASCOT acceptable format and searched using the Mascot database search algorithm. The allowed variable modifications for database searching were oxidation of methionines.

Western Blot Analysis of rhMFG-E8

Purified rhMFG-E8 proteins were electrophoretically fractionated on a 10-20% Tris-HCl gel under reducing conditions, transferred to a 0.45-μm nitrocellulose membrane, and blocked with 5% nonfat dry milk in phosphate-buffered saline. Afterward, the membrane was incubated with 1:1000 polyclonal antibody to human MFG-E8 (R & D Systems, Minneapolis, Minn.) overnight at 4° C. The blots were then incubated with horseradish peroxidase-linked anti-rabbit immunoglobulin G (1:10000, Cell Signaling Technology, Beverly, Mass.) for 1 h at room temperature. A chemiluminescent peroxidase substrate (ECL, Amersham Biosciences, Piscataway, N.J.) was applied according to the manufacturer's instructions, and the membranes were exposed briefly to radiography film.

### Phagocytosis Assay

This assay was conducted as previously described (Miksa, et al., 2009a). Briefly, freshly collected peritoneal macrophages from normal adult Sprague-Dawley rats were cultured in Dulbecco's Modified Eagle's Medium (DMEM; GIBCO Life Technologies, Carlsbad, Calif.) containing 10% heat-inactivated exosome-free fetal bovine serum (FBS), 10 mM 4-(2-hydroxyethyl)-1-piperazineethanesulfonic acid (HEPES), 100 U/ml penicillin and 100 mg/ml streptomycin at 37° C. in a humidified atmosphere containing 5% CO₂. Cells were plated at a density of 2.5×10⁴/well in a 16-well chamber slide (Nunc International, Rochester, N.Y.). For all experiments, cells were kept at 80-90% confluence. Freshly collected thymocytes were cultured at a concentration of 10⁷ cells/ml in RPMI substituted with 10% heat-inactivated FBS, 10 mM HEPES, 100 U/ml penicillin, 100 mg/ml streptomycin, and 0.1 μM dexamethasone for 16-24 h at 37° C. and 5% CO₂. This produced ˜100% apoptotic cells as assessed by annexin V/propidium iodide (PI) staining and analyzed by FACS. After being washed twice with Hank's balanced salt solution (HBSS, GIBCO), the apoptotic thymocytes were resuspended in OPTI-MEM (GIBCO) and incubated with or without rhMFG-E8 (0.5 μg/ml) or rmMFG-E8 (0.5 μg/ml) for 30 min. Then the cells were incubated with 20 ng/ml pHrodo-Se (Invitrogen) for 30 min. After washing, the cells were fed to cultured macrophages at the ratio of 4:1 (apoptotic cells/macrophages) for 1.0 h. Then, adherent macrophages were washed twice with PBS and incubated with FITC-anti-rat CD11b/c (OX42; BD pharmingen) for 20 min. This staining provided a homogenous surface staining of macrophages and was useful to distinguish the surface of macrophages during analysis. The cells were then fixed with 1% paraformaldehyde for 15 min at 4° C., transferred to PBS and kept at 4° C. until analysis by fluorescent microscopy using a Nikon Eclipse E600 florescent microscope (Japan). The number of apoptotic cells and of macrophages that engulfed apoptotic cells were expressed as a ratio of apoptotic cells/macrophages (phagocytosis index).

### Animal Model of Sepsis

Male Sprague-Dawley rats (275-325 g) were housed in a temperature-controlled room on a 12-h light/dark cycle and fed a standard Purina rat chow diet. Prior to the induction of sepsis, rats were fasted overnight, but allowed water ad libitum. Rats were anesthetized with isoflurane inhalation and the ventral neck, abdomen and groin were shaved and washed with 10% povidone iodine. Cecal ligation and puncture (CLP) was performed as previously described (Wu, et al., 2007b; Cui, et al., 2004; Wu, et al., 2007a). Briefly, a 2-cm midline abdominal incision was performed. The cecum was exposed, ligated just distal to the ileocecal valve to avoid intestinal obstruction, punctured twice with an 18-gauge needle, squeezed slightly to allow a small amount of fecal matter to flow from the holes, and then returned to the abdominal cavity, following which the abdominal incision was closed in layers. Immediately after CLP, a femoral vein were cannulated with a PE-50 tubing under anesthesia (isoflurane inhalation). The animal received a bolus injection of rhMFG-E8 (20 μg/kg BW) in a volume of 1-ml normal saline via the femoral venous catheter. Positive control animals received commercial rmMFG-E8 (20 μg/kg BW). Vehicle-treated animals received a non-specific human plasma protein (i.e., human albumin) at the time of CLP. Sham-operated animals (i.e., control animals) underwent the same procedure with the exception that the cecum was neither ligated nor punctured. The animals were resuscitated with 3 ml/100 g BW normal saline subcutaneously immediately after surgery. The animals were then returned to their cages. All experiments were performed in accordance with the National Institutes of Health guidelines for the use of experimental animals. This project was approved by the Institutional Animal Care and Use Committee (IACUC) of The Feinstein Institute for Medical Research.

### Results

### Determination of Thymocyte Apoptosis

Thymocyte apoptosis was assessed by annexin V/propidium iodide (PI) staining and Western blot analysis of cleaved caspase-3 protein expression. Briefly, the fresh thymus was harvested at 20 h after CLP or sham operation. Thymocytes were isolated as described previously (Miksa, et al., 2009b). The cells were stained using the Annexin V Fluos staining kit (Boehringer Mannheim, Indianapolis, Ind.) according to the manufacturer's instruction and analyzed by flow cytometry with FACSCalibur (BD Biosciences). The annexin V⁺-PI⁻ cells were considered as apoptotic cells. Cleaved caspase-3 protein expression was measured by Western blot analysis similar to the method for rhMFG-E8 protein analysis, as described above. Specific antibodies against cleaved caspase-3 protein (Cell Signaling, Danvers, Mass.) were used. β-Actin was acid as the loading control.

### Determination of Serum Levels of Lactate and IL-6

Serum concentrations of lactate were determined by using the assay kit according to the manufacturer's instructions (Pointe Scientific, Lincoln Park, Mich.). Serum levels of IL-6 were measured using a commercially available enzyme-linked immunosorbent assay (ELISA) kit (BioSource International, Camarillo, Calif.) according to the manufacturer's instruction.

### Survival Study

In additional groups of animals, vehicle (human albumin), rhMFG-E8 or rmMFG-E8 (20 μg/kg BW) was administered immediately after CLP as described above. At 20 h after CLP, the gangrenous cecum was surgically excised and the peritoneal cavity was irrigated twice with 20 ml warm, sterile saline solution. The abdominal incision was then closed in layers, and rats received 3 ml/100 g BW saline subcutaneously. The animals were then returned to their cages and allowed food and water ad libitum. The charges in survival were monitored for 10 days.

### Statistical Analysis

All data are expressed as means±SE and compared by one-way analysis of variance (ANOVA). When the ANOVA was significant, post-hoc testing of differences between groups was performed using Student-Newman-Keuls method. The survival rate was estimated by Kaplan-Meier method and compared by the log-rank test. A P value<0.05 was considered statistically significant.

Expression and Purification of rhMFG-E8

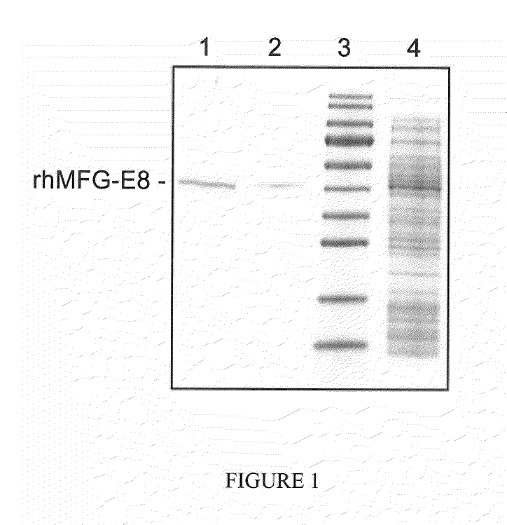

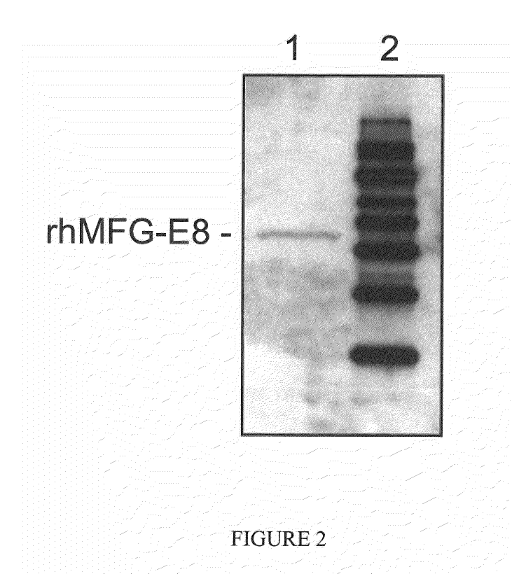

Using the E. coli system, rhMFG-E8 was successfully expressed and purified. The SDS-PAGE analysis showed a single band at approximately 46 kDa (FIG. 1). The purity of rhMFG-E8 is over 99% according to SDS-PAGE method (FIG. 1). The endotoxin level in the recombinant protein sample was not detectable as measured by Limulus Amebocyte Lysate method (data not shown). Western blot analysis showed that purified rhMFG-E8 was immunoreactive for a specific anti-human MFG-E8 antibodies (FIG. 2). Amino acid sequence analysis by LC-MS/MS showed that the purified protein was identified as human MFG-E8 with more than 95% confidence.

rhMFG-E8 Increased the Phagocytosis of Apoptotic Cells in Vitro

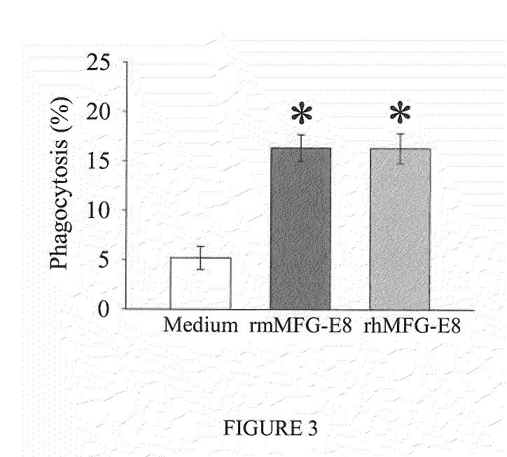

Using peritoneal macrophages isolated from normal rats, rhMFG-E8 (0.5 μg/ml) was shown to markedly increase peritoneal macrophages' phagocytosis of apoptotic thymocytes as compared to medium control (P<0.05, FIG. 3). Moreover, rhMFG-E8 is as effective as commercial rmMFG-E8 in the rat (FIG. 3). Thus, the purified rhMFG-E8 effectively increases the clearance of apoptotic cells in vitro.

rhMFG-E8 Reduced Apoptosis and Tissue Injury in a Rat Model of Sepsis

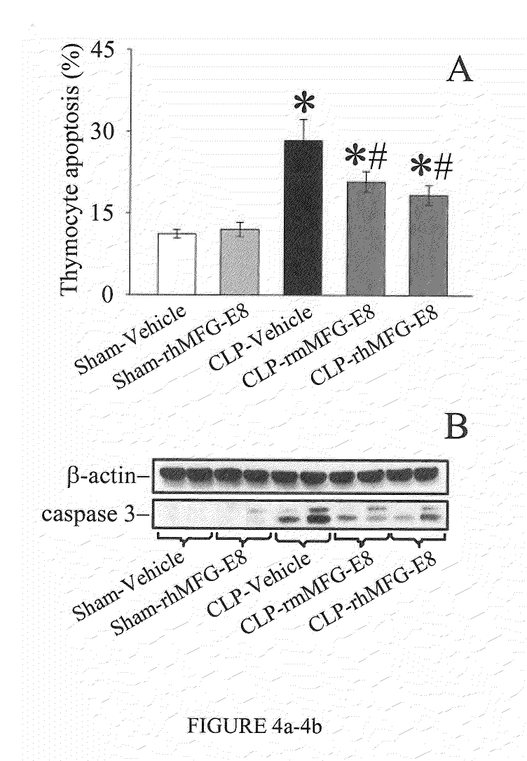

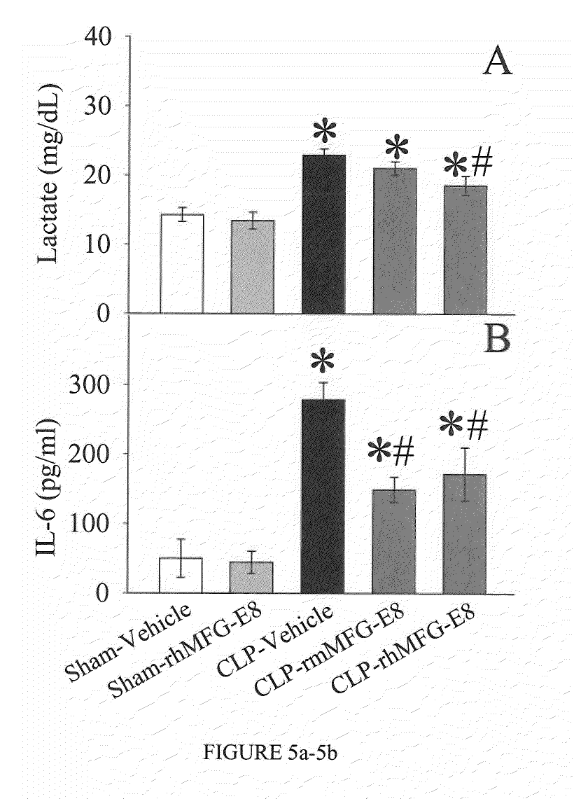

To determine the biological activity of the newly-expressed rhMFG-E8 in vivo, its effect was tested in a rat model of CLP. As shown in FIG. 4A, thymocyte apoptosis increased by relative 153% at 20 h after CLP in vehicle-treated animals. Administration of rmMFG-E8 or rhMFG-E8 decreased sepsis-induced thymocyte apoptosis by relative 27% and 35%, respectively (P<0.05). However, rhMFG-E8 had no effect on thymocyte apoptosis in sham-operated animals. These findings were confirmed by protein levels of cleaved caspase 3 (an indictor of cell apoptosis) in the thymus (FIG. 4B). Serum levels of lactate, a marker for systemic hypoxia, increased by 60% at 20 h after CLP. Administration of rhMFG-E8 decreased lactate levels by 19% (P<0.05, FIG. 5A). Similarly, serum levels of IL-6, an organ injury indicator as well as a marker for inflammation, increased by 457% at 20 h after CLP. Administration of rmMFG-E8 or rhMFG-E8 decreased IL-6 levels by 46% and 38% respectively (P<0.05, FIG. 5B).

rhMFG-E8 Decreased Sepsis-Induced Mortality in Rats

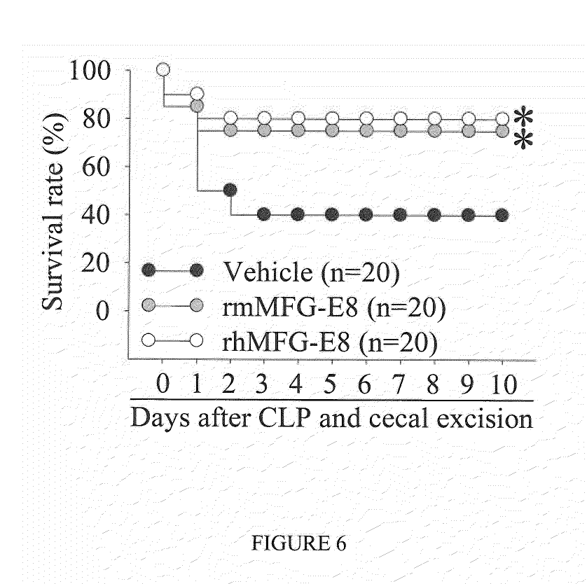

To determine the long-term effect of rhMFG-E8 is sepsis, a 10-day survival study was conducted. As shown in FIG. 6, the survival rate after CLP and cecal excision in vehicle (albumin) treated animals was 50% at day 2, and decreased to 40% at days 3-10. Administration of rmMFG-E8 or

### Discussion

Sepsis is a common, expensive, and frequently fatal condition. Although a great deal of preclinical and clinical trials have been carried out testing the efficacy and safety of various anti-sepsis agents (e.g., anti-cytokine and anti-endotoxin antibodies, steroids, antithrombin, and insulin, inhibition of apoptosis, etc.), those investigations have not resulted in the development of effective clinical treatment (Ferrer, et al., 2008; Strehlow, et al., 2006; Martin, et al., 2003; Guidet, et al., 2005). Apoptosis plays an important role in the pathobiology of sepsis (Hotchkiss and Nicholson, 2006; Remick, 2007; Lang and Matute-Bello, 2009; Pinheiro da and Nizet, 2009; Ward, 2008; Ayala, et al., 2008). Reduction of apoptosis by over-expressing the anti-apoptotic Bcl-2 protein or inhibiting pro-apoptotic molecules such as caspases, Fas-ligand, TNF-R, or TRAIL has been proven to be beneficial in septic animals (Hotchkiss, et al., 2005; Wesche, et al., 2005; Wesche-Soldato, et al., 2005; Ayala, et al., 2003; Zhou, et al., 2004; Bommhardt, et al., 2004). In addition to the increased incidence of apoptosis, the phagocytic function is impaired in sepsis (Gregory and Wing, 2002; Zhang, et al., 1998; Gutierrez-Fernandez, et al., 1989; Angle, et al., 2000). Previous studies have shown that downregulation of MFG-E8 is responsible for the reduced phagocytosis of apoptotic cell in sepsis (Miksa, et al., 2008; Miksa, et al., 2009c). Administration of rat MFG-E8 containing exosomes or rmMFG-E8 increases phagocytosis of apoptotic cells, reduces proinflammatory cytokines, and improves survival in a rat model of sepsis (Miksa, et al., 2008; Miksa, et al., 2009c). The biologic effect of this molecules has been confirmed using the MFG-E8 knockout animal model (Miksa, et al., 2009c). Similarly, Bu et al. has shown that sepsis-triggered intestinal injury was associated with a downregulation of intestinal MFG-E8 and treatment with rmMFG-E8 promoted mucosal healing in septic mice (Bu, et al., 2007). Thus, MFG-E8 appears to be a leading candidate for treating septic patients.

Human MFG-E8 shares only 59%, 57%, and 53% amino acid (aa) sequence identity with porcine, rat, and mouse MFG-E8, respectively (blast.ncbi.nlm.nih.gov). In order to move this technology into clinical development, human MFG-E8 is required. However, the extremely high cost of commercial human MFG-E8 (using murine myeloma cell line by R & D Systems) limits its further development. In the current study, rhMFG-E8 was successfully expressed and purified using an E. coli system at a much lower cost (>95% less expensive). The human MFG-E8 gene is located on chromosome 15q25 and is composed of eight exons. Human MGF-E8 protein is synthesized as a 387 as precursor that contains a 23 as signal sequence and a 364 aa mature region. The protein expressed in this study is the mature molecule of human MFG-E8 (i.e., Leu24-Cys387) with an N-terminal 6 His tag. Native MFG-E8 is a glycoprotein. Since rhMFG-E8 was expressed in an E. coli system, it has no glycosylation. As demonstrated by this study, E. coli-derived rhMFG-E8 is as effective as the rmMFG-E8 expressed in the murine myeloma cell line (R & D). E. coli-derived rhMFG-E8 markedly increased peritoneal macrophages' phagocytosis of apoptotic thymocytes and reduced thymocyte apoptosis and plasma levels of lactate and IL-6 at 20 h after CLP. Most importantly, administration of E. coli-derived rhMFG-E8 improved the survival rate after CLP. Apparently, glycosylation may not be essential for the biological function of MFG-E8.

The mature molecule of human MFG-E8 contains four N-linked glycosylation sites, an amino-terminal EGF like domain, plus C1 and C2 Ig-like domains which are related to discoidin 1 and homologous to those of human coagulation factors V and VIII (Couto, et al., 1996; Taylor, et al., 1997). The EGF like domain contains the “RGD-motif” (the amino acid sequence: Arg-Gly-Asp), which is strategically placed in a hairpin loop between two antiparallel beta strands (Couto, et al., 1996; Taylor, et al., 1997). In this way, the EGF-like domain serves as a scaffold for the RGD sequence, which is proposed to promote cell adhesion by binding cell surface integrin receptors, such as αᵥβ₃ or αᵥβ₅ (Akakura, et al., 2004; Zullig and Hengartner, 2004; Ait-Oufella, et al., 2007). The coagulation factor V/VIII like domains bind to phosphatidylserine (PS) exposed on the surface of apoptotic cells (Veron, et al., 2005). Binding of MFG-E8 to PS on apoptotic cells opsonizes them for a complete engulfment by macrophages via αᵥβ₃- or αᵥβ₅-integrins. Without MFG-E8, full engulfment and the removal of apoptotic cells cannot be completed (Hanayama, et al., 2004). Apoptosis has been considered as an orderly process of cell suicide that does not elicit inflammation (Fadok, et al., 1998). However, recent discoveries have shown that apoptotic cells eventually undergo secondary necrosis and stimulate an inflammatory response if they are not removed by phagocytosis (Bell, et al., 2006; Scaffidi, et al., 2002). The lack of clearance of apoptotic B-cells in the spleen potentially leads to autoimmune diseases (Hanayama, et al., 2002; Hanayama, et al., 2004). Similar phenomena were also reported in the acute inflammatory environment such as sepsis (Hotchkiss, et al., 2003; Miksa et al., 2009c). In a recent study, pre-treatment of animals with apoptotic splenocytes worsens the outcome of sepsis (Hotchkiss, et al., 2003), pointing out the detrimental effect of apoptotic cells in the septic organism. The current study confirms the importance of apoptotic cell clearance in the pathogenesis of sepsis.

### Example II

### Treatment of Cerebral Ischemia with Recombinant Human MFG-E8

### Materials and Methods

### Experimental Animals

Male Sprague-Dawley rats (300-350 g), purchased from Charles River Laboratories (Wilmington, Mass.) were used in this study. The rats were housed under standard conditions (room temperature, 22° C., 12/12-h light/dark cycle) with regular access to standard Purina rat chow and water. The animals were allowed at least 5 days to acclimate under these conditions before being used for experiments. All animal experiments were performed in accordance with the National Institutes of Health guidelines for the use of experimental animals. This protocol was approved by the Institutional Animal Care and Use Committee (IACUC) of the Feinstein Institute for Medical Research.

### Model of Cerebral Ischemia

Rats were fasted overnight but had access to water ad libitum before induction of cerebral ischemia. Permanent focal cerebral ischemia was induced by middle cerebral artery occlusion (MCAO) as previously described (Cheyuo et al., 2011; Zeng et al. 2010), with few modifications. Briefly, anesthesia was induced with 3.5% isoflurane and subsequently maintained by intravenous boluses of pentobarbital, not exceeding 30 mg/kg BW. Body temperature was maintained between 36.5° C. and 37.5° C. using a rectal temperature probe and a heating pad (Harvard Apparatus, Holliston, Mass.). The right common carotid artery (CCA) was exposed through a ventral midline neck incision and was carefully dissected free from the vagus nerve. The external carotid artery was then dissected and ligated. The internal carotid artery (ICA) was isolated and carefully separated from the adjacent vagus nerve, and the pterygopalatine artery was dissected and temporally occluded with a microvascular clip. Next, the CCA was ligated and an arteriotomy made just proximal to the bifurcation. A 2.5 cm length of 3-0 poly-L6 lysine coated monofilament nylon suture with a rounded tip was inserted through the arteriotomy into the ICA and advanced to the middle cerebral artery (MCA) origin to cause occlusion. Occlusion of the MCA was ascertained by inserting the suture to a predetermined length of 19-20 mm from the carotid bifurcation and feeling for resistance as the rounded suture tip approaches the proximal anterior cerebral artery which is of a relatively narrower caliber. The cervical wound was then closed in layers. One hour post-MCAO each rat was given an infusion of 1 ml saline as vehicle or 160 μg/kg BW rhMFG-E8 as treatment. Rats were then allowed to recover from anesthesia in a warm and quiet environment. The intraluminal suture was left in-situ and rats allowed unrestricted access to food and water until 24 h post-operatively when they were sacrificed. Brain tissues were rapidly collected for various anaylses. For histopathology, immunohistochemistry and terminal deoxynucleotidyl transferase dUTP nick end labeling (TUNEL), rat brains were transcardially perfused with ice-cold normal saline followed by 4% paraformaldehyde before removal. Brain samples were then paraffinembedded and sectioned.

### Assessment of Brain MFG-E8 Levels After Cerebral Ischemia

To establish a physiological basis for the use of MFG-E8 in the treatment of cerebral ischemia, changes in brain MFG-E8 levels after cerebral ischemia were assessed by Western blot. Briefly, protein obtained from brain homogenates of vehicle and sham groups were fractionated on a Bis-Tris gel and transferred to a 0.22 μm nitrocellulose membrane. Blots were blocked with 10% milk in Tris-buffered saline containing 0.1% vol/vol Tween 20 (TBST) and incubated with goat anti-MFG-E8 polyclonal IgG (1:100 in 10% BSA in TBST). After the incubation with horseradish peroxidase-labeled donkey anti-goat IgG (Santa Cruz Biotechnology Inc., Santa Cruz, Calif.) in 5% milk in TBST, and subsequently washing with TBST, bands were detected by chemiluminescence (GE Healthcare Biosciences, Piscataway, N.J.). The band densities were normalized by β-actin using the Bio-Rad imaging system.

Administration of rhMFG-E8

rhMFG-E8 was used as treatment in this study. rhMFG-E8 was expressed in-house using an E. coli system. The Ex-M0438-B01 expression clone for 6xHis-human MFG-E8 was purchased from GeneCopoeia, Inc (Germantown, Md.). The dose of rhMFG-E8 used in this study was chosen empirically based on previous experiments on dose-response effects of MFG-E8 in a sepsis model, which found the most efficacious dose to be 160 μg/kg BW (unpublished data).

### Assessment of Neurological Deficit

Neurological deficits were determined at 24 h post-MCAO using a battery of sensorimotor and reflex behavioral tests as outlined in Table 1. Prior to MCAO, animals were trained in the various sensorimotor and reflex behavioral tasks for two days. A combined neuroscore was calculated as a summation of the scores in sensorimotor and reflex behavioral tasks. Full details of these tests have been described elsewhere (Flierl et al. 2009; Kawamata et al. 1996; Markgraf et al. 1992).

### Assessment of Infarct Volume

Infarct size was determined as previously described (Lu et al. 2010). Rats were euthanized under anesthesia at 24 h post-MCAO. The brains were rapidly removed and sectioned coronally into 2 mm-thick slices which were incubated in 2% triphenyl tetrazolium chloride at 37° C. for 30 min and then immersed in 10% formula overnight. The pale-appearing infarcted areas, as well as areas of the hemispheres were digitally analysed using NIH Image J software. The infarct volume and volumes of the hemispheres in each slice were calculated as the area multiplied by 2. The total infarct volume and hemispheric volumes for each rat brain were calculated as summation of the individual slices. An edema index was calculated by dividing the total volume of the right hemisphere (ischemic side) by the total volume of the left hemisphere (non-ischemic side). The actual infarct volume adjusted for edema was calculated by dividing the infarct volume by the edema index, and expressed as percentage of the total brain volume.

### Histopathological Assessment

10 μm thick paraffin-embedded sections were stained with hematoxylin and eosin (H & E). The H & E stained slides were examined under bright field microscopy at 400× original magnification (Nikon Eclipse Ti microscope, Japan) for basophilic neurons, with purple-blue cytoplasm, and eosinophilic neurons, with pink cytoplasm, which are classified as intact neurons and necrotic neurons respectively (Ozden et al. 2011). Six images from six random visual fields were taken per slide. Quantification of intact neurons was performed while blinded to treatment allocations and functional outcomes. The average intact neuron count for each slide was expressed as intact neurons per 40× high power field.

### Determination of Cerebral Interleukin-6 Levels

Interleukin-6 (IL-6) levels in brain tissue lysates from the ipsilateral cerebral cortex were quantified by using commercially obtained enzyme-linked immunosorbent assay (ELISA) kits specific for IL-6 (BD BioSciences, San Jose, Calif.). The assay was carried out according to the instructions provided by the manufacturer.

### Determination of Cerebral TNF-α and Myeloperoxidase (MPO) Levels by Immunohistochemistry

6 μm paraffin sections of brain tissue were de-waxed and rehydrated, followed by microwave antigen retrieval procedure. Endogenous peroxidase and nonspecific binding sites were blocked using 2% H2O2 in 60% methanol and 3% normal goat serum respectively. The sections were then incubated with the following primary antibodies at room temperature for 2 h: rabbit anti-TNF-α polyclonal IgG (1:100, Millipore, Temecula, Calif.), and rabbit anti-MPO polyclonal IgG (1:100, Abcam, Cambridge, Mass.). The sections were then reacted with biotinylated anti-rabbit IgG, Vectastain ABC and DAB reagents (Vector Labs, Burlingame, Calif.). The immunohistochemical reaction was examined under light microscopy at 400× original magnification (Nikon E600 microscope, Japan). Neutrophils appeared as small, round, MPO-staining cells. Six images from six random fields in the penumbra of each slide were obtained. The average number of neutrophils was determined by NIH ImageJ particle analysis and expressed as neutrophils per 40× high power field.

### Determination of ICAM-1 Gene Expression

Total RNA extracted from cerebral cortex by Tri-Reagent (Molecular Research Center, Cincinnati, Ohio) was reverse transcribed into cDNA and real-time PCR performed as previously described (Wu et al. 2009b). Briefly, ICAM-1 gene expression was determined from cDNA using murine leukemia virus reverse transcriptase in an Applied Biosystems 7300 real-time PCR system (Applied Biosystems, Foster City, Calif.). Expression amount of rat glyceraldehyde-3-phosphate dehydrogenase (GAPDH) mRNA was used for normalization of each sample. Relative expression of mRNA was calculated by the 2-ΔΔCt method, and results expressed as fold change with respect to control. The following rat primers were used: ICAM-1(NM_{—}012967.1); Forward: 5′ CGA GTG GAC ACA ACT GGA AG 3′ (SEQ ID NO:5), Reverse: 5′ CGC TCT GGG AAC GAA TAC AC 3′ (SEQ ID NO:6).

### Western Blot for Peroxisome Proliferator-Activated Receptor-γ (PPAR-γ), Bcl-2 and Bax

Protein from homogenates of the ipsilateral cerebral cortex were fractionated on Bis-Tris gel and transferred to nitrocellulose membrane. Nitrocellulose blots were blocked in 5% milk in TBST (Tris-buffered saline Tween 20) and incubated overnight at 4° C. with the following rabbit polyclonal antibodies: anti-PPAR-γ (1:1000, Cayman Chemical, Ann Arbor, Mich.), anti-Bcl-2, and anti-Bax (1:500, Santa Cruz Biotechnology; Santa Cruz, Calif.). After reacting blots with HRP-labeled goat anti-rabbit IgG, protein bands were detected by chemiluminescence (GE Healthcare Biosciences, Piscataway, N.J.). The band densities were normalized by β-actin using the Bio-Rad imaging system.

### TUNEL Assay

6 μm paraffin sections of brain tissue were de-waxed and rehydrated, permeabilized with proteinase K, and then reacted with a green fluorescent-tagged enzyme solution (Roche Diagnostics, Indianapolis, Ind.). The slides were then washed with TBS, mounted with Vectashield medium with propidium iodide (Vector labs, Burlingame, Calif.) and examined under a fluorescence microscope (Nikon E600 microscope, Japan). Apoptotic cells appeared green fluorescent, and the nuclei which appeared red fluorescent confirmed the nuclear location of TUNEL products. Eight images were obtained from eight random visual fields in the penumbra of each slide at 200× original magnification. The average number of TUNEL positive cells for each slide was quantified by NIH ImageJ particle analysis and expressed as TUNEL positive cells per 2× high power field.

### Statistical Analysis

All data are expressed as means±SE and compared Student's ttest for two groups, and analysis of variance (ANOVA) and the Student-Newman-Keuls method for multiple groups. Differences in values were considered significant at p<0.05.

### Results

### Cerebral MFG-E8 Levels are Reduced by Cerebral Ischemia

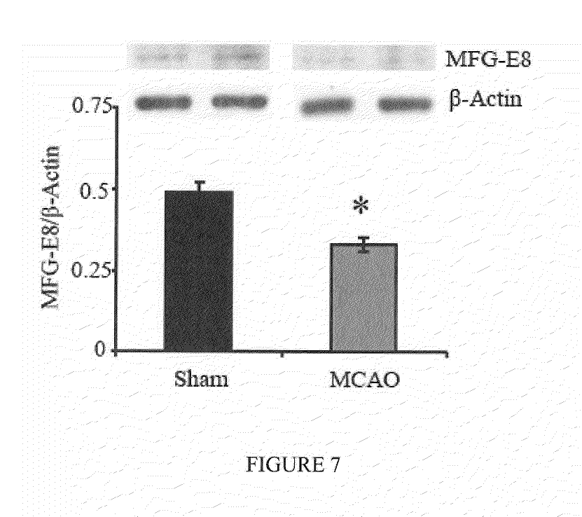

To determine whether permanent cerebral ischemia altered brain MFG-E8 levels, cerebral MFGE8 protein levels were measured at 24 h post-MCAO. As shown in FIG. 7, MCAO decreased cerebral MFG-E8 levels by 32.7% compared with sham (p<0.05).

rhMFG-E8 Treatment Improve Neurological Function

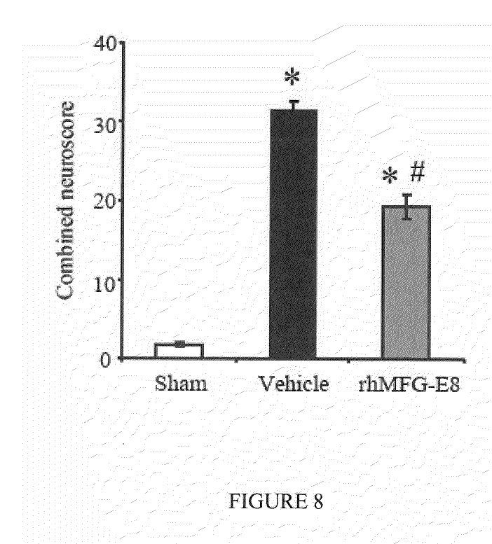

MCAO induced sensorimotor and reflex behavioral deficits compared with baseline neurological function in sham animals. As shown as FIG. 8, rhMFG-E8 treatment reduced the neurological deficits by 38.7% at 24 h post-MCAO compared with the vehicle group (p<0.05).

rhMFG-E8 Decreases Infarct Size and Neuronal Necrosis

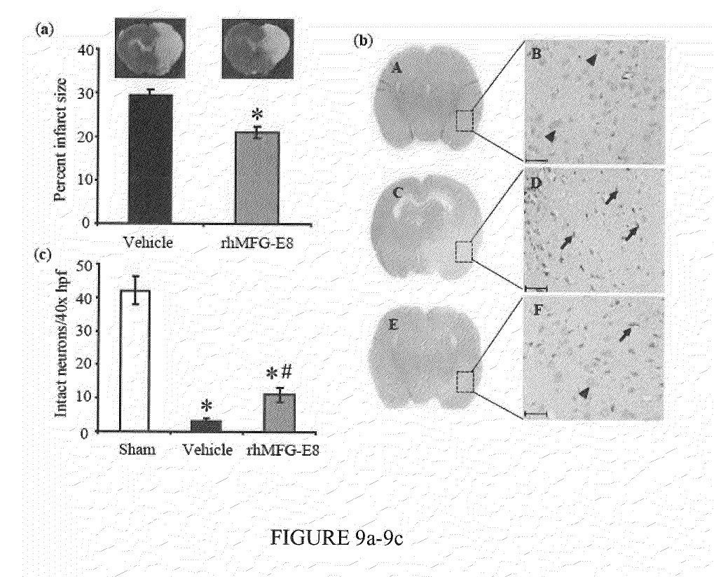

In the vehicle group, 24 hours of cerebral ischemia by MCAO caused infarction of 29.3% of the ipsilateral cerebral hemisphere. rhMFG-E8 treatment decreased the infarct size by 28.3% compared with vehicle (p<0.05, FIG. 9a). Twenty four hours of focal cerebral ischemia by MCAO resulted in profound neuronal necrosis, appearing as eosinophilic neurons on hematoxylin-eosin staining (FIG. 9b). Treatment with rhMFG-E8 protected neurons from necrosis resulting in a 267% increase in number of intact basophilic neurons compared to vehicle (p<0.05, FIG. 9c).

rhMFG-E8 Suppresses Inflammation in Cerebral Ischemia

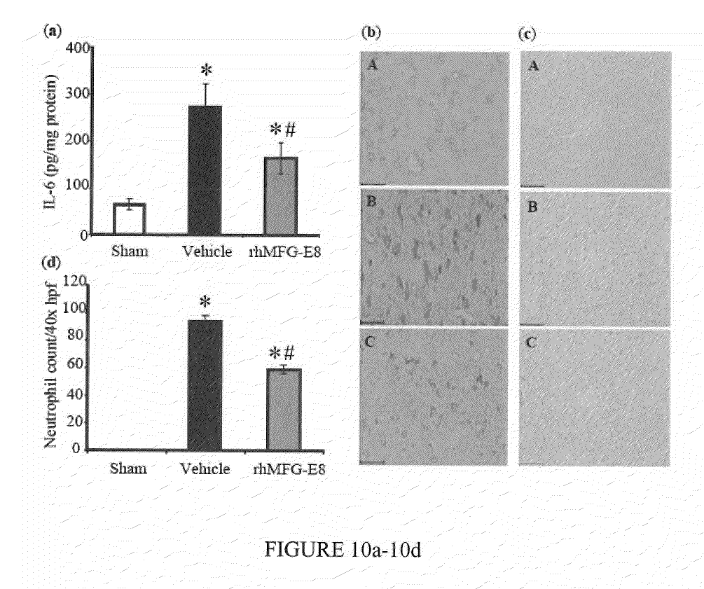

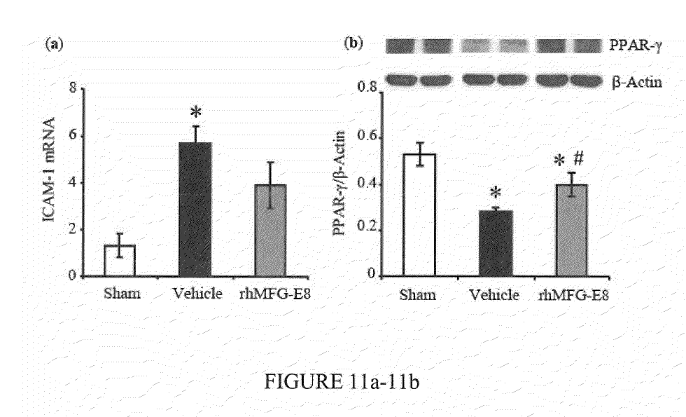

Compared with sham animals, MCAO resulted in elevations of cerebral IL-6 levels by 321% and 154% in vehicle and rhMFG-E8 treated animals respectively. Treatment with rhMFG-E8 decreased IL-6 levels by 39.6% compared with vehicle (p<0.05, FIG. 10a). Cerebral TNF-α levels were also increased by MCAO. rhMFG-E8 treated animals had less expression of TNF-α on immunohistochemistry compared with vehicle animals (FIG. 10b). An assessment of cerebral neutrophil infiltration in part of the inflammatory response showed that rhMFG-E8 treatment also decreased the number of infiltrated neutrophils compared with vehicle (FIG. 10c). In fact, FIG. 10d shows that rhMFG-E8 decreased the neutrophil infiltration by 37.2% compared with vehicle (p<0.05). Expression of ICAM-1, an adhesion molecule involved in neutrophil infiltration (Wiessner et al. 2009), was upregulated in vehicle group compared with sham (p<0.05 vs. Sham, FIG. 11a). Treatment with rhMFG-E8 decreased ICAM-1 gene expression by 31.6%, even though this downregulation was not significant compared with vehicle (FIG. 11a). MCAO downregulated the ligand-inducible transcription factor, PPAR-γ, in vehicle and rhMFG-E8 treated animals compared with sham. rhMFG-E8 treatment increased PPAR-γ levels by 39.3% compared with the vehicle group (p<0.05, FIG. 11b). PPAR-γ inhibits the expression of early inflammatory response genes including cytokines such as IL-6 (Yu et al. 2008). Thus, the mechanism of rhMFG-E8 anti-inflammatory effects may in part be due to upregulation of PPAR-γ.

rhMFG-E8 Inhibits Apoptosis in Cerebral Ischemia

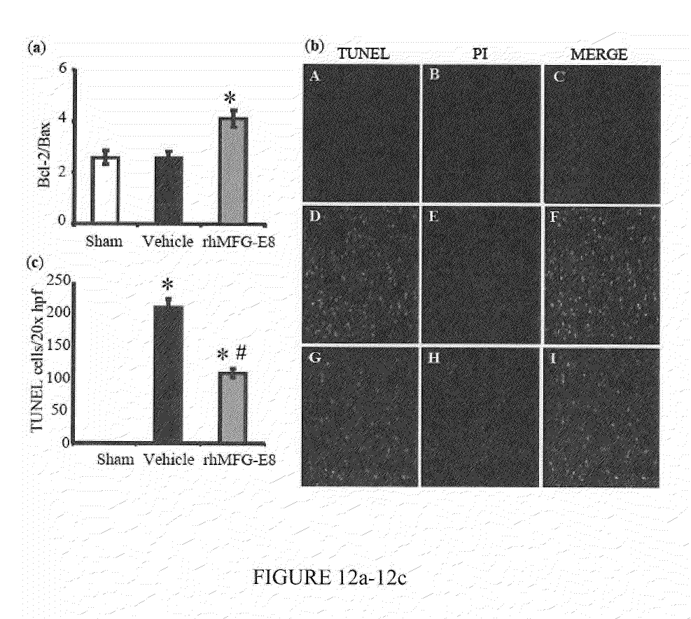

As shown in FIG. 12a, rhMFG-E8 treatment caused a 36.6% increase in Bcl2/Bax ratio compared with vehicle (p<0.05). TUNEL staining of brain sections showed fewer positive cells in rhMFG-E8 treated animals compared with vehicle (FIG. 12b). rhMFG-E8 treatment decreased the number of TUNEL positive cells by 48.2% compared with vehicle (p<0.05, FIG. 12c).

TABLE 1
Sensorimotor and reflex behavioral tests for assessment of neurological deficits

| SENSORIMOTOR | | REFLEXES AND BEHAVIORS | |
|---|---|---|---|
| Task | Score | Task | Score |

| Hemiplegia/Hemiparesis | | Postural reflex | | | |
|---|---|---|---|---|---|
| Resists change in position | 0 | Extension | | | 0 |
| Inability to resist | 1 | Flexion | | | 1 |
| Walking on the floor | | Righting reflex | | | |
| Walks straight | 0 | Forceful righting | | | 0 |
| Inability to walk straight | 1 | Strong righting | | | 1 |
| Inability to walk | 2 | Weak righting | | | 2 |
| Prostration | 3 | Attempts but unable to right | | | 3 |
| Walking on beam | | No attempt at righting | | | 4 |
| 1.0 cm wide | 0 | Limb placing reflex (all limbs) | | | |
| 2.5 cm wide | 1 | Visual-anterior | | | |
| 4.0 cm wide | 2 | Visual-sideways | | immediate | 0 |
| 6.0 cm wide | 3 | Tactile-anterior | {close oversize brace} | ≦2 s | 1 |
| Beam Balance Test | | Tactile-sideways | | ≧2 s | 2 |
| All four paws on top of beam | 1 | Proprioceptive | | | |

| | | | |
|---|---|---|---|
| Paws on side of beam | 2 | Seeking behavior | |
| One or two limbs off beam | 3 | Intact | 0 |
| Three or four limbs off beam | 4 | Lost | 1 |
| Attempts but falls off beam | 5 | Circle exit test (3 minutes) | |
| Drapes/hangs/fall of beam | 6 | Exit | 0 |
| No balance attempt, falls off | 7 | Inability to exit | 1 |

### Discussion

The MFG-E8 gene, which in humans is located at the chromosomal position 15q25 (Collins et al. 1997), is ubiquitously expressed in various tissues, including the brain, in mammals (Aziz et al. 2009; Hanayama et al. 2004; Larocca et al. 1991). MFG-E8 plays physiological roles in cell-cell interaction through the binding of αᵥβ_{3/5} integrin receptors (Raymond et al. 2010). Under pathological conditions, MFG-E8 has been shown to promote clearance of apoptotic cells by binding phosphatidylserine exposed on the apoptotic cells and simultaneously engaging the αᵥβ_{3/5} integrin receptor on macrophages (Hanayama et al. 2002). Apoptotic cells undergo secondary necrosis leading to the release of damage associated molecular pattern (DAMP) molecules which promote inflammation and tissue injury (Miksa et al. 2006). Promotion of apoptotic clearance has been shown to be beneficial in various brain disease. MFG-E8 levels are reduced in Alzheimer's disease, and decreased MFG-E8 mediated clearance of apoptotic neurons and amyloid β-peptides have been shown to play pathogenetic roles in Alzheimer's disease (Boddaert et al. 2007). The macrophage scavenger receptor A (CD204), which acts similar to MFG-E8 promoting macrophage clearance of apoptotic cells, has been shown to be neuroprotective in focal cerebral ischemia (Lu et al. 2010). However, the effects of MFG-E8 on cerebral ischemia have not been previously investigated. This study has established for the first time a beneficial role of MFG-E8 in focal cerebral ischemia, attributable, at least in part, to suppression of inflammation and apoptosis.

Cerebral ischemia downregulated cerebral MFG-E8 expression at 24 hours after onset of ischemia. Intravenous administration of exogenous rhMFG-E8 one hour after the ischemia reduced infarct size and improved neurological function. Histopathological examination also showed that rhMFG-E8 treatment protected neurons in the penumbra against necrosis. Inflammation (Schilling et al. 2003) and apoptosis (Broughton et al. 2009) play crucial roles in tissue damage during cerebral ischemia. The anti-inflammatory effects of rhMFG-E8 treatment in cerebral ischemia included suppression of cytokine (IL-6 and TNF-α) release, ICAM-1 expression, and cerebral neutrophil infiltration. Upregulation of the ligand-inducible transcription factor, PPAR-γ, may be responsible for the inhibition of cytokine release following treatment with rhMFG-E8. Focal cerebral ischemia by MCAO suppressed PPAR-γ levels. Treatment with rhMFG-E8 attenuated the ischemia-induced downregulation of PPAR-γ compared with vehicle. PPAR-γ is known to suppress NF-κB mediated cytokine production through a variety of mechanisms collectively termed transrepression (Ricote and Glass 2007). PPAR-γ agonists such as the thiazolidinediones have shown neuroprotection in cerebral ischemia (Wang et al. 2009). The finding that rhMFG-E8 mediated upregulation of PPAR-γ together with suppression of cytokine release is consistent with the work of Zhang et al who showed that the PPAR-γ agonist, pioglitazone, suppresses NF-κB signaling in permanent focal cerebral ischemia, resulting in neuroprotection (Zhang et al. 2011).

rhMFG-E8 treatment was also demonstrated to inhibit apoptosis in cerebral ischemia, rhMFG-E8 treatment decreased TUNEL staining in the penumbra and also increased Bcl-2/Bax ratio. Upregulation of the Bcl-2/Bax ratio may be an integrinmediated effect of rhMFG-E8 treatment. The MFG-E8 receptor, αᵥβ₃, has been shown to increase Bcl-2 transcription through a focal adhesion kinase (FAK)-dependent activation of the PI3K-Akt pathway (Matter and Ruoslahti 2001). The anti-apoptotic effect of rhMFG-E8 may further be explained by the upregulation of PPAR-γ. Wu et al showed that PPAR-γ overexpression inhibited apoptosis in cerebral ischemia. Knockdown of PPAR-γ using small interfering RNA abrogated the anti-apoptotic effects of PPAR-γ (Wu et al. 2009a).

The CX3X chemokine, fractalkine, stimulates the release of MFG-E8 from microglial cells (Leonardi-Essmann et al. 2005). The neuroprotective effects of MFG-E8 under conditions of hypoxia/ischemia are further supported by in vitro studies by other investigators using fractalkine. Noda et al showed that fractalkine decreased excitotoxicity by stimulating microglial clearance of apoptotic neurons. Treatment of the microglial cells with anti-MFG-E8 neutralizing antibodies abolished the microglial clearance of necrotic neurons and diminished the neuroprotection (Noda et al. 2011). Similarly, Kranich et al also showed that MFG-E8 protects against neurotoxicity in a model of prion disease (Kranich et al. 2010).

In summary, this first assessment of the role of rhMFG-E8 in cerebral ischemia has shown that rhMFG-E8 suppresses inflammation and apoptosis in a permanent focal cerebral ischemia model and that rhMFG-E8 is a novel neuroprotective agent for cerebral ischemia.

### REFERENCES

Aida Y and Pabst M J (1990) Removal of endotoxin from protein solutions by phase separation using Triton X-114, J Immunol Methods 132:191-195.
Ait-Oufella H, Kinugawa K, Zoll J, Simon T, Boddaert J, Heeneman S, Blanc-Brude O, Barateau V, Potteaux S, Merval R, Esposito B, Teissier E, Daemen M J, Leseche G, Boulanger C, Tedgui A and Mallat Z (2007) Lactadherin deficiency leads to apoptotic cell accumulation and accelerated atherosclerosis in mice. Circulation 115:2168-2177.
Akakura S, Singh S, Spataro M, Akakura R, Kim J I, Albert M L, and Birge R B (2004) The opsonin MFG-E8 is a ligand for the alphavbeta5 integrin and triggers DOCK180-dependent Rac1 activation for the phagocytosis of apoptotic cells. Exp Cell Res 292:403-416.
Andersen M H, Berglund L, Rasmussen J T, Petersen T E (1997) Bovine PAS-6/7 binds alpha v beta 5 integrins and anionic phospholipids through two domains. Biochemistry 36:5441-6.
Angle N, Cabello-Passini R, Hoyt D B, Loomis W H, Shreve A, Namiki S and Junger W G (2000) Hypertonic saline infusion: can it regulate human neutrophil function? Shock 14:503-508.
Angus D C, Linde-Zwirble W T, Lidicker J, Clermont G, Carcillo J and Pinsky M R (2001) Epidemiology of severe sepsis in the United States: analysis of incidence, outcome, and associated costs of care. Crit Care Med 29:1303-1310.
Antonsson B (2004) Mitochondria and the Bcl-2 family proteins in apoptosis signaling pathways. Mol Cell Biochem 256-257:141-155.
Ayala A, Lomas J L, Grutkoski P S and Chung S (2003) Fas-ligand mediated apoptosis in severe sepsis and shock. Scand J Infect Dis 35:593-600.
Ayala A, Perl M, Venet F, Lomas-Neira J, Swan R and Chung C S (2008) Apoptosis in sepsis: mechanisms, clinical impact and potential therapeutic targets. Curr Pharm Des 14:1853-1859.
Aziz M M, Ishihara S, Mishima Y, Oshima N, Moriyama I, Yuki T, Kadowaki Y, Rumi M A, Amano Y, Kinoshita Y (2009) MFG-E8 attenuates intestinal inflammation in murine experimental colitis by modulating osteopontin-dependent alphavbeta3 integrin signaling. J Immunol 182:7222-7232.
Bell C W, Jiang W, Reich C F, III and Pisetsky D S (2006) The extracellular release of HMGB1 during apoptotic cell death. Am J Physiol Cell Physiol 291:C1318-C1325.
Boddaert J, Kinugawa K, Lambert J C, Boukhtouche F, Zoll J, Merval R, Blanc-Brude O, Mann D, Berr C, Vilar J, Garabedian B, Journiac N, Charue D, Silvestre J S, Duyckaerts C, Amouyel P, Mariani J, Tedgui A, Mallat Z (2007) Evidence of a role for lactadherin in Alzheimer's disease. Am J Pathol 170:921-929.
Bommhardt U, Chang K C, Swanson P E, Wagner T H, Tinsley K W, Karl I E and Hotchkiss R S (2004) Akt decreases lymphocyte apoptosis and improves survival in sepsis. J Immunol 172:7583-7591.
Broughton B R, Reutens D C, Sobey C G (2009) Apoptotic mechanisms after cerebral ischemia. Stroke 40:e331-e339.
Bu H F, Zuo X L, Wang X, Ensslin M A, Koti V, Hsueh W, Raymond A S, Shur B D and Tan X D (2007) Milk fat globule-EGF factor 8/lactadherin plays a crucial role in maintenance and repair of murine intestinal epithelium. J Clin Invest 117:3673-3683.
Cheyno C, Wu R, Zhou M, Jacob A, Coppa G, Wang P (2011) Ghrelin suppresses inflammation and neuronal nitric oxide synthase in focal cerebral ischemia via the vagus nerve. Shock 35:258-265.
Collins C, Nehlin J O, Stubbs J D, Kowbel D, Kuo W L, Parry G (1997) Mapping of a newly discovered human gene homologous to the apoptosis associated-murine mammary protein, MFG-E8, to chromosome 15q25. Genomics 39:117-118.
Couto J R, Taylor M R, Godwin S G, Ceriani R L and Peterson J A (1996) Cloning and sequence analysis of human breast epithelial antigen BA46 reveals an RGD cell adhesion sequence presented on an epidermal growth factor-like domain. DNA Cell Biol 15:281-286.
Cui T, Miksa M, Wu R, Komura H, Zhou M, Dong W, Wang Z, Higuchi S, Chaung W, Blau S A, Marini C P, Ravikumar T S, Wang P (2010) Milk fat globule epidermal growth factor 8 attenuates acute lung injury in mice after intestinal ischemia and reperfusion. Am J Respir Crit Care Med 181:238-246.
Cui X, Wu R, Zhou M, Simms H H and Wang P (2004) Different expression of cytochrome P450 isoforms in the lungs of septic animals. Crit Care Med 32:1186-1191.
Dirnagl U, Iadecola C, Moskowitz M A (1999) Pathobiology of ischemic stroke: an integrated view, Trends Neurosci 22:391-397.
Fadok V A, Bratton D L, Konowal A, Freed P W, Westcott J Y and Henson P M (1998) Macrophages that have ingested apoptotic cells in vitro inhibit proinflammatory cytokine production through autocrine/paracrine mechanisms involving TGF-beta, PGE2, and PAF. J Clin Invest 101:890-898.
Ferrer R, Artigas A, Levy M M, Blanco J, Gonzalez-Diaz G, Garnacho-Montero J, Ibanez J, Palencia E, Quintana M and de la Torre-Prados M V (2008) Improvement in process of care and outcome after a multicenter severe sepsis educational program in Spain. JAMA 299:2294-2303.
Flierl M A, Stahel P F, Beauchamp K M, Morgan S J, Smith W R, Shohami E (2009) Mouse closed head injury model induced by a weight-drop device. Nat Protoc 4:1328-1337.
Fuller A D, Van Eldik L J, (2008) MFG-E8 regulates microglial phagocytosis of apoptotic neurons. J Neuroimmune Pharmacol 3:246-256.
Gregory S H and Wing E J (2002) Neutrophil-Kupffer cell interaction: a critical component of host defenses to systemic bacterial infections. J Leukoc Biol 72:239-248.
Guidet B, Aegerter P, Gauzit R, Meshaka P and Dreyfuss D (2005) Incidence and impact of organ dysfunctions associated with sepsis. Chest 127:942-951.
Gutierrez-Fernandez J, Maroto M C, Piedrola G and Zamora E (1989) Dysfunction of the mononuclear phagocytic system in sepsis. APMIS 97:441-446.
Hanayama R, Tanaka M, Miwa K, Shinohara A, Iwamatsu A, Nagata S (2002) Identification of a factor that links apoptotic cells to phagocytes. Nature 417:182-187.
Hanayama R, Tanaka M, Miyasaka K, Aozasa K, Koike M, Uchiyama Y, Nagata S (2004) Autoimmune disease and impaired uptake of apoptotic cells in MFG-E8-deficient mice. Science 304:1147-1150.
Hotchkiss R S, Chang K C, Grayson M H, Tinsley K W, Dunne B S, Davis C G, Osborne D F and Karl I E (2003) Adoptive transfer of apoptotic splenocytes worsens survival, whereas adoptive transfer of necrotic splenocytes improves survival in sepsis. Proc Natl Acad Sci U S A 100:6724-6729.
Hotchkiss R S, Coopersmith C M and Karl I E (2005) Prevention of lymphocyte apoptosis—a potential treatment of sepsis? Clin Infect Dis 41 Suppl 7:S465-S469.
Hotchkiss R S and Nicholson D W (2006) Apoptosis and caspases regulate death and inflammation in sepsis. Nat Rev Immunol 6:813-822.
Kawamata T, Alexis N E, Dietrich W D, Finklestein S P (1996) Intracisternal basic fibroblast growth factor (bFGF) enhances behavioral recovery following focal cerebral infarction in the rat. J Cereb Blood Flow Metab 16:542-547.
Kranich J, Krautler N J, Falsig J, Ballmer B, Li 5, Hutter G, Schwarz P, Moos R, Julius C, Miele G, Aguzzi A (2010) Engulfment of cerebral apoptotic bodies controls the course of prion disease in a mouse strain-dependent manner. J Exp Med 207:2271-2281.
Lang J D end Matute-Bello G (2009) Lymphocytes, apoptosis and sepsis: making the jump from mice to humans. Crit Care 13:109.
Larocca D, Peterson J A, Urrea R, Kuniyoshi J, Bistrain A M, Ceriani R L (1991) A Mr 46,000 human milk fat globule protein that is highly expressed in human breast tumors contains factor VIII-like domains. Cancer Res 51:4994-4998.
Leonardi-Essmann F, Emig M, Kitamura Y, Spanagel R, Gebicke-Haerter P J (2005) Fractalkine-upregulated milk-fat globule EGF factor-8 protein in cultured rat microglia. J Neuroimmunol 160:92-101.
Li J, Wang H, Mason J M, Levine J, Yu M, Ulloa L, Czura C J, Tracey K J and Yang H (2004) Recombinant HMGB1 with cytokine-stimulating activity. J Immunol Methods 289:211-223.
Lu C, Hua F, Liu L, Ha T. Kalbfleisch J, Schweitzer J, Kelley J, Kao R, Williams D, Li C (2010) Scavenger receptor class-A has a central role in cerebral ischemiareperfusion injury. J Cereb Blood Flow Metab 30:1972-1981.
Martin G S, Mannino D M, Eaton S and Moss M (2003) The epidemiology of sepsis in the United States from 1979 through 2000. N Engl J Med 348:1546-1554.
Markgraf C G, Green E J, Hurwitz B E, Morikawa E, Dietrich W D, McCabe P M, Ginsberg M D, Schneiderman N (1992) Sensorimotor and cognitive consequences of middle cerebral artery occlusion in rats. Brain Res 575:238-246.
Matter M L, Ruoslahti E (2001) A signaling pathway from the alpha5beta1 and alpha(v)beta3 integrins that elevates bcl-2 transcription. J Biol Chem 276:27757-27763.
Miksa M, Amin D, Wu R, Jacob A, Zhou M, Dong W, Yang W L, Ravikumar T S and Wang P (2008) Maturation-induced down-regulation of MFG-E8 impairs apoptotic cell clearance and enhances endotoxin response. Int J Mol Med 22:743-748.
Miksa M, Komura H, Wu R, Shah K G and Wang P (2009a) A novel method to determine the engulfment of apoptotic cells by macrophages using pHrodo succinimidyl ester. J Immunol Methods 342:71-77.
Miksa M, Komura H, Wu R, Shah K G and Wang P (2009b) A novel method to determine the engulfment of apoptotic cells by macrophages using succinimidyl ester. J Immunol Methods 342:71-77.
Miksa M, Wu R, Dong W, Das P, Yang D, Wang P (2006) Dendritic cell-derived exosomes containing milk fat globule epidermal growth factor-factor VIII attenuate proinflammatory responses in sepsis. Shock 25:586-593.
Miksa M, Wu R, Dong W, Komura H, Amin D, Ji Y, Wang Z, Wang H, Ravikumar T S, Tracey K J and Wang P (2009c) Immature dendritic cell-derived exosomes rescue septic animals via milk fat globule epidermal growth factor VIII. J Immunol 183:5983-5990.
Miyasaka K, Hanayama R, Tanaka M and Nagata S (2004) Expression of milk fat globule epidermal growth factor 8 in immature dendritic cells for engulfment of apoptotic cells. Eur J Immunol 34:1414-1422.
Noda M, Doi Y, Liang J, Kawanokuchi J, Sonobe Y, Takeuchi H, Mizuno T, Suzumura A (2011). Fractalkine attenuates excito-neurotoxicity via microglial clearance of damaged neurons and antioxidant enzyme heme oxygenase-1 expression. J Biol Chem 286:2308-2319.
Oshima K, Aoki N, Kato T, Kitajima K and Matsuda T (2002) Secretion of a peripheral membrane protein, MFG-E8, as a complex with membrane vesicles. Eur J Biochem 269:1209-1218.
Ozden H, Durmaz R, Kanbak G, Uzuner K, Aral E, Kartkaya K, Kabay S C, Atasoy M A (2011) Erythropoietin prevents nitric oxide and cathepsin-mediated neuronal death in focal brain ischemia. Brain Res 1370:185-193.
Pinheiro da S F and Nizet V (2009) Cell death during sepsis: integration of disintegration in the inflammatory response to overwhelming infection. Apoptosis 14:509-521.
Raymond A S, Elder B, Ensslin M, Shur B D (2010) Loss of SED1/MFG-E8 results in altered luminal physiology in the epididymis. Mol Reprod Dev 77:550-563.
Remick D G (2007) Pathophysiology of sepsis. Am J Pathol 170:1435-1444.
Ricote M, Glass CK (2007) PPARs and molecular mechanisms of transrepression. Biochim Biophys Acta 1771:926-935.
Scaffidi P, Misteli T and Bianchi M E (2002) Release of chromatin protein HMGB1 by necrotic cells triggers inflammation. Nature 418:191-195.
Schilling M, Besselmann M, Leonhard C, Mueller M, Ringelstein E B, Kiefer R (2003) Microglial activation precedes and predominates over macrophage infiltration in transient focal cerebral ischemia: a study in green fluorescent protein transgenic bone marrow chimeric mice. Exp Neurol 183:25-33.
Shao C, Novakovic V A, Head J F, Seaton B A, Gilbert G E (2008) Crystal structure of lactadherin C2 domain at 1.7 A resolution with mutational and computational analyses of its membrane-binding motif. J Biol Chem 283:7230-7241.
Strehlow M C, Emond S D, Shapiro N I, Pelletier A J and Camargo C A, Jr. (2006) National study of emergency department visits for sepsis, 1992 to 2001. Ann Emerg Med 48:326-331.
Stubbs J D, Lekutis C, Singer K L, Bui A, Yuzuki D, Srinivasan U, Parry G (1990) cDNA cloning of a mouse mammary epithelial cell surface protein reveals the existence of epidermal growth factor-like domains linked to factor VIII-like sequences. Proc Natl Acad Sci U S A 87:8417-8421.
Taylor M R, Couto J R, Scallan C D, Ceriani R L, Peterson J A (1997) Lactadherin (formerly BA46), a membrane-associated glycoprotein expressed in human milk and breast carcinomas, promotes Arg-Gly-Asp (RGD)-dependent cell adhesion. DNA Cell Biol 16:861-869.
Thery C, Regnault A, Garin J, Wolfers J, Zitvogel L, Ricciardi-Castagnoli P, Raposo G and Amigorena S (1999) Molecular characterization of dendritic cell-derived exosomes. Selective accumulation of the heat shock protein hsc73. J Cell Biol 147:599-610.
Veron P, Segura E, Sugano G, Amigorena S and Thery C (2005) Accumulation of MFG-E8/lactadherin on exosomes from immature dendritic cells. Blood Cells Mol Dis 35:81-88.
Wang C X, Ding X, Noor R, Pegg C, He C, Shuaib A (2009) Rosiglitazone alone or in combination with tissue plasminogen activator improves ischemic brain injury in an embolic model in rats. J Cereb Blood Flow Metab 29:1683-1694.
Ward P A (2008) Sepsis, apoptosis and complement. Biochem Pharmacol 76:1383-1388.
Wesche D E, Lomas-Neira J L, Perl M, Chung C S and Ayala A (2005) Leukocyte apoptosis and its significance in sepsis and shock. J Leukoc Biol 78:325-337.
Wesche-Soldato D E, Chung C S, Lomas-Neira J, Doughty L A, Gregory S H and Ayala A (2005) In vivo delivery of caspase-8 or Fas siRNA improves the survival of septic mice Blood 106:2295-2301.
Wiessner R, Eisold S, Linnebacher M, Bunger C, Nizze H, Wacke R, Benz S, Schareck W, Klar E (2009) Up-regulation of ICAM-1 during cold ischemia triggers early neutrophil infiltration in human pancreas allograft reperfusion. Transplant Proc 41:3622-3627.
Wu J S, Cheung W M, Tsai Y S, Chen Y T, Fong W H, Tsai H D, Chen Y C, Liou J Y, Shyue S K, Chen J J, Chen Y E, Maeda N, Wu K K, Lin T N (2009a) Ligand-activated peroxisome proliferator-activated receptor-gamma protects against ischemia cerebral infarction and neuronal apoptosis by 14-3-3 epsilon upregulation. Circulation 119:1124-1134.
Wu K, Dong W, Cui X, Zhou M, Simms H H, Ravikumar T S and Wang P (2007a) Ghrelin down-regulates proinflammatory cytokines a sepsis through activation of the vagus nerve. Ann Surg 245:480-486.
Wu R, Dong W, Zhou M, Zhang F, Marini C P, Ravikumar T S and Wang P (2007b) Ghrelin attenuates sepsis-induced acute lung injury and mortality in rats. Am J Respir Crit Care Med 176:805-813.
Wu R, Zhou M, Dong W, Ji Y, Miksa M, Marini C P, Ravikumar T S, Wang P (2009b) Ghrelin hyporesponsiveness contributes to age-related hyperinflammation in septic shock. Ann Surg 250:126-133.
Yolken R H, Peterson J A, Vonderfecht S L, Fouts E T, Midthun K and Newburg D S (1992) Human milk mucin inhibits rotavirus replication and prevents experimental gastroenteritis. J Clin Invest 90:1984-1991.
Yu J H, Kim K H, Kim H (2008) SOCS 3 and PPAR-gamma ligands inhibit the expression of IL-6 and TGF-beta1 by regulating JAK2/STAT3 signaling in pancreas. Int J Biochem Cell Biol 40:677-688.
Zeng H K, Wang Q S, Deng Y Y, Jiang W Q, Fang M, Chen C B, Jiang X (2010) A comparative study on the efficacy of 10% hypertonic saline and equal volume of 20% mannitol in the treatment of experimentally induced cerebral edema in adult rats. BMC Neurosci 11:153.
Zhang H L, Xu M, Wei C, Qin A P, Lin C F, Hong L Z, Zhao X Y, Liu J, Qin Z H (2011) Neuroprotective effects of pioglitazone in a rat model of permanent focal cerebral ischemia are associated with peroxisome proliferator-activated receptor gammamediated suppression of nuclear factor-kappaB signaling pathway. Neuroscience 176:381-395.
Zhang P, Bagby G J, Stoltz D A, Summer W R and Nelson S (1998) Enhancement of peritoneal leukocyte function by granulocyte colony-stimulating factor in rats with abdominal sepsis. Crit Care Med 26:315-321.
Zhou M, Simms H H and Wang P (2004) Adrenomedullin and adrenomedullin binding protein-1 attenuate vascular endothelial cell apoptosis in sepsis. Ann Surg 240:321-330.
Zullig S and Hengartner M O (2004) Cell biology. Tickling macrophages, a serious business. Science 304:1123-1124.
U.S. Patent Application Publication No. 2009/0297498, Milk Fat Globule Epidermal Growth Factor-Factor VIII And Sepsis, Wang, Ping, published Dec. 3, 2009.
PCT International Publication No. WO 2009/064448, Prevention and Treatment of Inflammation and Organ Injury after Ischemia/Reperfusion using MFG-E8, Wang, Ping, published May 22, 2009.

1. A method of preventing and/or treating cerebral ischemia in a subject comprising administering to the subject a milk fat globule epidermal growth factor-factor VIII (MFG-E8) in an amount effective to prevent and/or treat cerebral ischemia.

2. The method of claim 1, wherein the subject has cerebral ischemia.

3. The method of claim 1, wherein the subject is a patient at risk for cerebral ischemia.

4. The method of claim 1, wherein the cerebral ischemia is focal brain ischemia caused by a blood clot that occludes a cerebral blood vessel.

5. The method of claim 1, wherein the cerebral ischemia is global brain ischemia caused by reduced blood flow to the brain.

6. The method of claim 1, wherein administering MFG-E8 reduces cerebral level of interleukin-6 (IL-6).

7. The method of claim 1, wherein administering MFG-E8 reduces numbers of infiltrated neutrophils.

8. The method of claim 1, wherein administering MFG-E8 reduces cerebral inflammation and/or apoptosis.

9. The method of claim 1, wherein administering MFG-E8 reduces and/or prevents death of brain tissue.

10. The method of claim 1, wherein chance of survival of the subject is increased.

11. A method of preparing a pharmaceutical composition for preventing and/or treating cerebral ischemia, the method comprising formulating milk fat globule epidermal growth factor-factor VIII (MFG-E8) in a pharmaceutical composition in an amount effective to prevent and/or treat cerebral ischemia.

12. The method of claim 11, wherein the MFG-E8is a recombinant human MFG-E8 (rhMFG-E8).

13. The method of claim 12, wherein the rhMFG-E8 has an amino acid sequence that is at least 95% identical to human MFG-E8 (hMFG-E8) (SEQ ID NO:1).

14. The method of claim 12, wherein the rhMFG-E8 has an amino acid sequence that is at least 99% identical to human MFG-E8 (hMFG-E8) (SEQ ID NO:1).

15. The method of claim 12, wherein the rhMFG-E8 has an amino acid sequence that is identical to human MFG-E8 (hMFG-E8) (SEQ ID NO:1).

16. The method of claim 12, wherein the rhMFG-E8 is non-glycosylated.

17-60. (canceled)


SEQUENCE LISTING (6 sequences)

SEQ ID NO: 1 - protein, 364 aa, Homo sapiens
LDICSKNPCHNGGLCEEISQEVRGDVFPSYTCTCLKGYAGNHCETKCVEPLGMENGNIANSQIAASSVRVTFLGLQHWVPELARLNRAGMVNAWTPSSNDDNPWIQVNLLRRMWVTGVVTQGASRLASHEYLKAFKVAYSLNGHEFDFIHDVNKKHKEFVGNWNKNAVHVNLFETPVEAQYVRLYPTSCHTACTLRFELLGCELNGCANPLGLKNNSIPDKQITASSSYKTWGLHLFSWNPSYARLDKQGNFNAWVAGSYGNDQWLQVDLGSSKEVTGIITQGARNFGSVQFVASYKVAYSNDSANWTEYQDPRTGSSKIFPGNWDNHSHKKNLFETPILARYVRILPVAWHNRIALRLELLGC

SEQ ID NO: 2 - protein, 387 aa, Homo sapiens
MPRPRLLAALCGALLCAPSLLVALDICSKNPCHNGGLCEEISQEVRGDVFPSYTCTCLKGYAGNHCETKCVEPLGMENGNIANSQIAASSVRVTFLGLQHWVPELARLNRAGMVNAWTPSSNDDNPWIQVNLLRRMWVTGVVTQGASRLASHEYLKAFKVAYSLNGHEFDFIHDVNKKHKEFVGNWNKNAVHVNLFETPVEAQYVRLYPTSCHTACTLRFELLGCELNGCANPLGLKNNSIPDKQITASSSYKTWGLHLFSWNPSYARLDKQGNFNAWVAGSYGNDQWLQVDLGSSKEVTGIITQGARNFGSVQFVASYKVAYSNDSANWTEYQDPRTGSSKIFPGNWDNHSHKKNLFETPILARYVRILPVAWHNRIALRLELLGC

SEQ ID NO: 3 - protein, 463 aa, Mouse
MQVSRVLAALCGMLLCASGLFAASGDFCDSSLCLNGGTCLTGQDNDIYCLCPEGFTGLVCNETERGPCSPNPCYNDAKCLVTLDTQRGDIFTEYICQCPVGYSGIHCETETNYYNLDGEYMFTTAVPNTAVPTPAPTPDLSNNLASRCSTQLGMEGGAIADSQISASYVYMGFMGLQRWGPELARLYRTGIVNAWHASNYDSKPWIQVNLLRKMRVSGVMTQGASRAGRAEYLKTFKVAYSLDGRKFEFIQDESGGDKEFLGNLDNNSLKVNMFNPTLEAQYIRLYPVSCHRGCTLRFELLGCELHGCLEPLGLKNNTIPDSQMSASSSYKTWNLRAFGWYPHLGRLDNQGKINAWTAQSNSAKEWLQVDLGTQRQVTGIITQGARDFGHIQYVESYKVAHSDDGVQWTVYEEQGSSKVFQGNLDNNSHKKNIFEKPFMARYVRVLPVSWHNRITLRLELLGC

SEQ ID NO: 4 - DNA, 1095 bp, Homo sapiens
ctggatatctgttccaaaaacccctgccacaacggtggtttatgcgaggagatttcccaagaagtgcgaggagatgtcttcccctcgtacacctgcacgtgccttaagggctacgcgggcaaccactgtgagacgaaatgtgtcgagccactgggcatggagaatgggaacattgccaactcacagatcgccgcctcatctgtgcgtgtgaccttcttgggtttgcagcattgggtcccggagctggcccgcctgaaccgcgcaggcatggtcaatgcctggacacccagcagcaatgacgataacccctggatccaggtgaacctgctgcggaggatgtgggtaacaggtgtggtgacgcagggtgccagccgcttggccagtcatgagtacctgaaggccttcaaggtggcctacagccttaatggacacgaattcgatttcatccatgatgttaataaaaaacacaaggagtttgtgggtaactggaacaaaaacgcggtgcatgtcaacctgtttgagacccctgtggaggctcagtacgtgagattgtaccccacgagctgccacacggcctgcactctgcgctttgagctactgggctgtgagctgaacggatgcgccaatcccctgggcctgaagaataacagcatccctgacaagcagatcacggcctccagcagctacaagacctggggcttgcatctcttcagctggaacccctcctatgcacggctggacaagcagggcaacttcaacgcctgggttgcggggagctacggtaacgatcagtggctgcaggtggacctgggctcctcgaaggaggtgacaggcatcatcacccagggggcccgtaactttggctctgtccagtttgtggcatcctacaaggttgcctacagtaatgacagtgcgaactggactgagtaccaggaccccaggactggcagcagtaagatcttccctggcaactgggacaaccactcccacaagaagaacttgtttgagacgcccatcctggctcgctatgtgcgcatcctgcctgtagcctggcacaaccgcatcgccctgcgcctggagctgctgggctgttag

SEQ ID NO: 5 - DNA, 20 bp, artificial
  forward primer for ICAM-1
cgagtggacacaactggaag

SEQ ID NO: 6 - DNA, 20 bp, artificial
  reverse primer for ICAM-1
cgctctgggaacgaatacac